(19) United States
(12) Patent Application Publication
(10) Pub. No.: US 2023/0413806 A1
(43) Pub. Date: Dec. 28, 2023

(54) ENHANCEMENT OF ALLOGRAFT QUALITY BY POSTMORTEM DONOR REGENERATION

(71) Applicant: VENVALO GROUP LLC, Miami Beach, FL (US)

(72) Inventors: Thomas E. ICHIM, San Diego, CA (US); Kalina O'CONNOR, San Diego, CA (US); Boris N. REZNIK, Miami Beach, FL (US)

(21) Appl. No.: 18/339,135

(22) Filed: Jun. 21, 2023

Related U.S. Application Data
(60) Provisional application No. 63/354,830, filed on Jun. 23, 2022.

(51) Int. Cl. A01N 1/02; C12N 5/074; C12N 5/0786

(52) U.S. Cl. CPC A01N 1/0205; C12N 5/0696; C12N 5/0645; C12N 2500/02; C12N 2500/05; C12N 2501/113; C12N 2501/12; C12N 2501/231

(57) ABSTRACT

Described are means, methods and compositions of matter useful for enhancing quality of organ transplants by induction of postmortem regeneration. The disclosure provides administration of regenerative cells and/or factors in a brain dead recipient whose body is maintained in a viable state by life supporting machinery.

### CROSS-REFERENCE TO RELATED APPLICATIONS

This application claims the benefit of U.S. Provisional Application Ser. No. 63/354,830, filed on Jun. 23, 2022, entitled “ENHANCEMENT OF ALLOGRAFT QUALITY BY POSTMORTEM DONOR REGENERATION,” which is hereby incorporated by reference in its entirety and for all purposes under 37 CFR 1.57.

### BACKGROUND

### Field of the Disclosure

The disclosure pertains to the field of organ transplantation, more specifically to the field of regeneration of organs. More particularly the disclosure relates to the field of increasing viability and quality of organs to increase the donor organ pool.

### Description of the Related Art

Organ transplantation has seen the lives of many patients with end stage organ failure. Currently there is a severe organ shortage that can be addressed by reducing the number of marginal donors. The disclosure seeks to accomplish this by providing means of in vivo organ rejuvenation.

### SUMMARY

For purposes of summarizing the invention and the advantages achieved over the prior art, certain objects and advantages of the invention have been described herein. Of course, it is to be understood that not necessarily all such objects or advantages may be achieved in accordance with any particular embodiment of the invention. Thus, for example, those skilled in the art will recognize that the invention may be embodied or carried out in a manner that achieves or optimizes one advantage or group of advantages as taught herein without necessarily achieving other objects or advantages as may be taught or suggested herein.

In one embodiments, a method of increasing the quality of organs for transplantation is described. The method may include, for example, a) obtaining a brain-dead patient; b) maintaining viability of said brain dead patient by one or more life supporting technologies; c) administering to said patient one or more regenerative cell populations; and d) harvesting said organs.

In some embodiments, the regenerative cell may be a stem cell such as a pluripotent stem cell or a mesenchymal stem cell. In some embodiments, the pluripotent stem cells may be selected from a group of cells. The group of cells may comprise: a) inducible pluripotent stem cells; b) somatic cell nuclear transfer derived stem cells; c) embryonic stem cells; and d) parthenogenic derived stem cells. In some embodiments, the pluripotent stem cells may be exposed to inflammatory stress before being provided to the brain dead patient. In some embodiments, the inflammatory stress may be exposure to a toll-like receptor.

In some embodiments, the inducible pluripotent stem cell of the group of cells may possess markers selected from a group comprising: CD10, CD13, CD44, CD73, CD90, PDGFr-alpha, PD-L2, and HLA-A,B,C. The inducible pluripotent stem cells may also possess an ability to undergo at least 40 doublings in culture while maintaining a normal karyotype upon passaging. In some embodiments, the said inducible pluripotent stem cells may also express OCT4.

In some embodiments, the parthenogenically derived stem cells of the group of cells may be generated by addition of a calcium flux inducing agent to activate an oocyte followed by enrichment of cells expressing markers selected from a group comprising of SSEA-4, TRA 1-60 and TRA 1-81. In some embodiments, the somatic cell nuclear transfer derived stem cells of the group of cells may possess a phenotype negative for SSEA-1 and positive for SSEA-3, SSEA-4, TRA-1-60, TRA-1-81, and alkaline phosphatase.

In some embodiments, the mesenchymal stem cell of the group of cells may be derived from tissue comprising a group selected from: a) Wharton's Jelly; b) bone marrow; c) peripheral blood; d) mobilized peripheral blood; e) endometrium; f) hair follicle; g) deciduous tooth; h) testicle; i) adipose tissue; j) skin; k) amniotic fluid; l) cord blood; m) omentum; n) muscle; o) amniotic membrane; o) periventricular fluid; and p) placental tissue. In some embodiments, the mesenchymal stem cells may express a marker or plurality of markers selected from a group comprising of: STRO-1, CD90, CD73, CD105, CD54, CD106, HLA-I markers, vimentin, ASMA, collagen-1, fibronectin, LFA-3, ICAM-1, PECAM-1, P-selectin, L-selectin, CD49b/CD29, CD49c/CD29, CD49d/CD29, CD61, CD18, CD29, thrombomodulin, telomerase, CD10, CD13, STRO-2, VCAM-1, CD146, and THY-1. In some embodiments, the mesenchymal stem cells may not express substantial levels of HLA-DR, CD117, and CD45. In some embodiments, the mesenchymal stem cells may express CD56. In some embodiments, the mesenchymal stem cell may be activated by exposure to a toll like receptor agonist.

In some embodiments, the said regenerative cells are monocytes. In some embodiments, the regenerative cells are monocytes that have been treated with interleukin-10. In some embodiments, the regenerative cells are monocytes that have been exposed to hypoxia. In some embodiments, the regenerative cells are monocytes that have been exposed to HGF-1. In some embodiments, the regenerative cells are monocytes that have been exposed to FGF-1. In some embodiments, the regenerative cells are monocytes that have been exposed to krypton.

All of these embodiments are intended to be within the scope of the invention herein disclosed. These and other embodiments of the present invention will become readily apparent to those skilled in the art from the following detailed description of the preferred embodiments, the invention not being limited to any particular preferred embodiment(s) disclosed.

### DETAILED DESCRIPTION

The illustrative embodiments described herein are not meant to be limiting. Other embodiments may be utilized, and other changes may be made, without departing from the spirit or scope of the subject matter presented. It will be readily understood that the aspects of the present disclosure can be arranged, substituted, combined, and designed in a wide variety of different configurations by a person of ordinary skill in the art, all of which are made part of this disclosure.

Reference in the specification to “one embodiment,” “an embodiment”, or “in some embodiments” means that a particular feature, structure, or characteristic described in connection with the embodiment is included in at least one embodiment of the invention. Moreover, the appearance of these or similar phrases throughout the specification does not necessarily mean that these phrases all refer to the same embodiment, nor are separate or alternative embodiments necessarily mutually exclusive. Various features are described herein which may be exhibited by some embodiments and not by others. Similarly, various requirements are described which may be requirements for some embodiments but may not be requirements for other embodiments.

The disclosure teaches in vivo regeneration techniques and protocols for post-mortem increasing the quality of donor organs by administration of regenerative cells in a brain dead patient. In one embodiment the disclosure provides administration of mesenchymal stem cells into a patient on life support who is brain dead. In some embodiments the disclosure provides means of concurrently stimulating regeneration of tissue as well as blood vessels surrounding said tissues.

For the practice of the disclosure, MSC are a type of stem cell utilized for inducing regeneration of the endometrium. “Mesenchymal stem cell” or “MSC” in some embodiments refers to cells that are (1) adherent to plastic, (2) express CD73, CD90, and CD105 antigens, while being CD14, CD34, CD45, and HLA-DR negative, and (3) possess ability to differentiate to osteogenic, chondrogenic and adipogenic lineage. Other cells possessing mesenchymal-like properties are included within the definition of “mesenchymal stem cell”, with the condition that said cells possess at least one of the following: a) regenerative activity; b) production of growth factors; c) ability to induce a healing response, either directly, or through elicitation of endogenous host repair mechanisms. As used herein, “mesenchymal stromal cell” or ore mesenchymal stem cell can be used interchangeably. Said MSCcan be derived from any tissue including, but not limited to, bone marrow, adipose tissue, amniotic fluid, endometrium, trophoblast-derived tissues, cord blood, Wharton jelly, placenta, amniotic tissue, derived from pluripotent stem cells, and tooth. In some definitions of “MSC”, said cells include cells that are CD34 positive upon initial isolation from tissue but are similar to cells described about phenotypically and functionally. As used herein, “MSC” may includes cells that are isolated from tissues using cell surface markers selected from the list comprised of NGF-R, PDGF-R, EGF-R, IGF-R, CD29, CD49a, CD56, CD63, CD73, CD105, CD106, CD140b, CD146, CD271, MSCA-1, SSEA4, STRO-1 and STRO-3 or any combination thereof, and satisfy the ISCT criteria either before or after expansion. Furthermore, as used herein, in some contexts, “MSC” includes cells described in the literature as bone marrow stromal stem cells (BMSSC), marrow-isolated adult multipotent inducible cells (MIAMI) cells, multipotent adult progenitor cells (MAPC), mesenchymal adult stem cells (MASCS), MultiStem®, Prochymal®, remestemcel-L, Mesenchymal Precursor Cells (MPCs), Dental Pulp Stem Cells (DPSCs), PLX cells, PLX-PAD, AlloStem®, Astrostem®, Ixmyelocel-T, MSC-NTF, NurOwn™, Stemedyne™-MSC, Stempeucel®, StempeucelCLI, StempeucelOA, HiQCell, Hearticellgram-AMI, Revascor®, Cardiorel®, Cartistem®, Pneumostem®, Promostem®, Homeo-GH, AC607, PDA001, SB623, CX601, AC607, Endometrial Regenerative Cells (ERC), adipose-derived stem and regenerative cells (ADRCs).

In one embodiment, the cells of the present disclosure are generally referred to as umbilical-derived cells (or UDCs). They also may sometimes be referred to more generally herein as postpartum-derived cells or postpartum cells (PPDCs). In addition, the cells may be described as being stem or progenitor cells, the latter term being used in the broad sense. The term derived is used to indicate that the cells have been obtained from their biological source and grown or otherwise manipulated in vitro (e.g., cultured in a growth medium to expand the population and/or to produce a cell line). The in vitro manipulations of umbilical stem cells and the unique features of the umbilicus-derived cells of the present disclosure are described in detail below.

Various terms are used to describe cells in culture. Cell culture refers generally to cells taken from a living organism and grown under controlled condition (“in culture” or “cultured”). A primary cell culture is a culture of cells, tissues, or organs taken directly from an organism(s) before the first subculture. Cells are expanded in culture when they are placed in a growth medium under conditions that facilitate cell growth and/or division, resulting in a larger population of the cells. When cells are expanded in culture, the rate of cell proliferation is sometimes measured by the amount of time needed for the cells to double in number. This is referred to as doubling time.

A conditioned medium is a medium in which a specific cell or population of cells has been cultured, and then removed. When cells are cultured in a medium, they may secrete cellular factors that can provide trophic support to other cells. Such trophic factors include, but are not limited to hormones, cytokines, extracellular matrix (ECM), proteins, vesicles, antibodies, and granules. The medium containing the cellular factors is the conditioned medium. In one specific embodiment of the disclosure, supernatant is collected from MSC selected for ability to suppress fibrosis. In other embodiments, MSC are chosen based on angiogenic activity. Said angiogencic activity is identified based on proteomic and other analysis of markers, proteins, and peptides that are correlated with enhanced ability to induce regeneration. In a specific embodiment the disclosure provides means of regenerating endometrium using said conditioned media. In some embodiments of the disclosure, the inventors interchangeably use the words “conditioned media” and “trophic factors”. Generally, a trophic factor is defined as a substance that promotes or at least supports, survival, growth, proliferation and/or maturation of a cell, or stimulates increased activity of a cell.

When referring to cultured vertebrate cells, the term senescence (also replicative senescence or cellular senescence) refers to a property attributable to finite cell cultures; namely, their inability to grow beyond a finite number of population doublings (sometimes referred to as Hayflick's limit). Although cellular senescence was first described using fibroblast-like cells, most normal human cell types that can be grown successfully in culture undergo cellular senescence. The in vitro lifespan of different cell types varies, but the maximum lifespan is typically fewer than 100 population doublings (this is the number of doublings for all the cells in the culture to become senescent and thus render the culture unable to divide). Senescence does not depend on chronological time, but rather is measured by the number of cell divisions, or population doublings, the culture has undergone. Thus, cells made quiescent by removing essential growth factors are able to resume growth and division when the growth factors are re-introduced, and thereafter carry out the same number of doublings as equivalent cells grown, continuously. Similarly, when cells are frozen in liquid nitrogen after various numbers of population doublings and then thawed and cultured, they undergo substantially the same number of doublings as cells maintained unfrozen in culture. Senescent cells are not dead or dying cells; they are actually resistant to programmed cell death (apoptosis), and have been maintained in their nondividing state for as long as three years. These cells are very much alive and metabolically active, but they do not divide. The nondividing state of senescent cells has not yet been found to be reversible by any biological, chemical, or viral agent.

As used herein, the term Growth Medium generally refers to a medium sufficient for the culturing of umbilicus-derived cells. In particular, one presently preferred medium for the culturing of the cells of the disclosure herein comprises Dulbecco's Modified Essential Media (also abbreviated DMEM herein). Particularly preferred is DMEM-low glucose (also DMEM-LG herein) (Invitrogen, Carlsbad, Calif.). The DMEM-low glucose is preferably supplemented with 15% (v/v) fetal bovine serum (e.g. defined fetal bovine serum, Hyclone, Logan Utah), antibiotics/antimycotics (preferably penicillin (100 Units/milliliter), streptomycin (100 milligrams/milliliter), and amphotericin B (0.25 micrograms/milliliter), (Invitrogen, Carlsbad, Calif.)), and 0.001% (v/v) 2-mercaptoethanol (Sigma, St. Louis Mo.). In some cases, different growth media are used, or different supplementations are provided, and these are normally indicated in the text as supplementations to Growth Medium.

Also relating to the present disclosure, the term standard growth conditions, as used herein refers to culturing of cells at 37.degree. C., in a standard atmosphere comprising 5% CO.sub.2. Relative humidity is maintained at about 100%. While foregoing the conditions are useful for culturing, it is to be understood that such conditions are capable of being varied by the skilled artisan who will appreciate the options available in the art for culturing cells, for example, varying the temperature, CO.sub.2, relative humidity, oxygen, growth medium, and the like.

Oct-4 (oct-3 in humans) is a transcription factor expressed in the pregastrulation embryo, early cleavage stage embryo, cells of the inner cell mass of the blastocyst, and embryonic carcinoma (“EC”) cells (Nichols, J. et al. (1998) Cell 95: 379-91), and is down-regulated when cells are induced to differentiate. The oct-4 gene (oct-3 in humans) is transcribed into at least two splice variants in humans, oct-3A and oct-3B. The oct-3B splice variant is found in many differentiated cells whereas the oct-3A splice variant (also previously designated oct-3/4) is reported to be specific for the undifferentiated embryonic stem cell. See Shimozaki et al. (2003) Development 130: 2505-12. Expression of oct-3/4 plays an important role in determining early steps in embryogenesis and differentiation. Oct-3/4, in combination with rox-1, causes transcriptional activation of the Zn-finger protein rex-1, which is also required for maintaining ES cells in an undifferentiated state (Rosfjord, E. and Rizzino, A. (1997) Biochem Biophys Res Commun 203: 1795-802; Ben-Shushan, E. et al. (1998) Mol Cell Biol 18: 1866-78).

In one embodiment MSC donor lots are generated from umbilical cord tissue. Means of generating umbilical cord tissue MSC have been previously published and are incorporated by reference [1-7]. The term “umbilical tissue derived cells (UTC)” refers, for example, to cells as described in U.S. Pat. Nos. 7,510,873, 7,413,734, 7,524,489, and 7,560,276. The UTC can be of any mammalian origin e.g. human, rat, primate, porcine and the like. In one embodiment of the disclosure, the UTC are derived from human umbilicus. umbilicus-derived cells, which relative to a human cell that is a fibroblast, a mesenchymal stem cell, or an iliac crest bone marrow cell, have reduced expression of genes for one or more of: short stature homeobox 2; heat shock 27 kDa protein 2; chemokine (C-X-C motif) ligand 12 (stromal cell-derived factor 1); elastin (supravalvular aortic stenosis, Williams-Beuren syndrome); Homo sapiens mRNA; cDNA DKFZp586M2022 (from clone DKFZp586M2022); mesenchyme homeobox 2 (growth arrest-specific homeobox); sine oculis homeobox homolog 1 (Drosophila); crystallin, alpha B; disheveled associated activator of morphogenesis 2; DKFZP586B2420 protein; similar to neuralin 1; tetranectin (plasminogen binding protein); src homology three (SH3) and cysteine rich domain; cholesterol 25-hydroxylase; runt-related transcription factor 3; interleukin 11 receptor, alpha; procollagen C-endopeptidase enhancer; frizzled homolog 7 (Drosophila); hypothetical gene BC008967; collagen, type VIII, alpha 1; tenascin C (hexabrachion); iroquois homeobox protein 5; hephaestin; integrin, beta 8; synaptic vesicle glycoprotein 2; neuroblastoma, suppression of tumorigenicity 1; insulin-like growth factor binding protein 2, 36 kDa; Homo sapiens cDNA FLJ12280 fis, clone MAMMA1001744; cytokine receptor-like factor 1; potassium intermediate/small conductance calcium-activated channel, subfamily N, member 4; integrin, beta 7; transcriptional co-activator with PDZ-binding motif (TAZ); sine oculis homeobox homolog 2 (Drosophila); KIAA1034 protein; vesicle-associated membrane protein 5 (myobrevin); EGF-containing fibulin-like extracellular matrix protein 1; early growth response 3; distal-less homeobox 5; hypothetical protein FLJ20373; aldo-keto reductase family 1, member C3 (3-alpha hydroxysteroid dehydrogenase, type II); biglycan; transcriptional co-activator with PDZ-binding motif (TAZ); fibronectin 1; proenkephalin; integrin, beta-like 1 (with EGF-like repeat domains); Homo sapiens mRNA full length insert cDNA clone EUROIMAGE 1968422; EphA3; KIAA0367 protein; natriuretic peptide receptor C/guanylate cyclase C (atrionatriuretic peptide receptor C); hypothetical protein FLJ14054; Homo sapiens mRNA; cDNA DKFZp564B222 (from clone DKFZp564B222); BCL2/adenovirus E1B 19 kDa interacting protein 3-like; AE binding protein 1; and cytochrome c oxidase subunit VIIa polypeptide 1 (muscle). In addition, these isolated human umbilicus-derived cells express a gene for each of interleukin 8; reticulon 1; chemokine (C-X-C motif) ligand 1 (melonoma growth stimulating activity, alpha); chemokine (C-X-C motif) ligand 6 (granulocyte chemotactic protein 2); chemokine (C-X-C motif) ligand 3; and tumor necrosis factor, alpha-induced protein 3, wherein the expression is increased relative to that of a human cell which is a fibroblast, a mesenchymal stem cell, an iliac crest bone marrow cell, or placenta-derived cell. The cells are capable of self-renewal and expansion in culture, and have the potential to differentiate into cells of other phenotypes.

Methods of deriving cord tissue mesenchymal stem cells from human umbilical tissue are provided. The cells are capable of self-renewal and expansion in culture, and have the potential to differentiate into cells of other phenotypes. The method comprises (a) obtaining human umbilical tissue; (b) removing substantially all of blood to yield a substantially blood-free umbilical tissue, (c) dissociating the tissue by mechanical or enzymatic treatment, or both, (d) resuspending the tissue in a culture medium, and (e) providing growth conditions which allow for the growth of a human umbilicus-derived cell capable of self-renewal and expansion in culture and having the potential to differentiate into cells of other phenotypes. Tissue can be obtained from any completed pregnancy, term or less than term, whether delivered vaginally, or through other routes, for example surgical Cesarean section. Obtaining tissue from tissue banks is also considered within the scope of the present disclosure.

The tissue is rendered substantially free of blood by any means known in the art. For example, the blood can be physically removed by washing, rinsing, and diluting and the like, before or after bulk blood removal for example by suctioning or draining. Other means of obtaining a tissue substantially free of blood cells might include enzymatic or chemical treatment.

Dissociation of the umbilical tissues can be accomplished by any of the various techniques known in the art, including by mechanical disruption, for example, tissue can be aseptically cut with scissors, or a scalpel, or such tissue can be otherwise minced, blended, ground, or homogenized in any manner that is compatible with recovering intact or viable cells from human tissue.

In a presently preferred embodiment, the isolation procedure also utilizes an enzymatic digestion process. Many enzymes are known in the art to be useful for the isolation of individual cells from complex tissue matrices to facilitate growth in culture. As discussed above, a broad range of digestive enzymes for use in cell isolation from tissue is available to the skilled artisan. Ranging from weakly digestive (e.g. deoxyribonucleases and the neutral protease, dispase) to strongly digestive (e.g. papain and trypsin), such enzymes are available commercially. A nonexhaustive list of enzymes compatable herewith includes mucolytic enzyme activities, metalloproteases, neutral proteases, serine proteases (such as trypsin, chymotrypsin, or elastase), and deoxyribonucleases. Presently preferred are enzyme activates selected from metalloproteases, neutral proteases and mucolytic activities. For example, collagenases are known to be useful for isolating various cells from tissues. Deoxyribonucleases can digest single-stranded DNA and can minimize cell-clumping during isolation. Enzymes can be used alone or in combination. Serine protease are preferably used in a sequence following the use of other enzymes as they may degrade the other enzymes being used. The temperature and time of contact with serine proteases must be monitored. Serine proteases may be inhibited with alpha 2 microglobulin in serum and therefore the medium used for digestion is preferably serum-free. EDTA and DNase are commonly used and may improve yields or efficiencies. Preferred methods involve enzymatic treatment with for example collagenase and dispase, or collagenase, dispase, and hyaluronidase, and such methods are provided wherein in certain preferred embodiments, a mixture of collagenase and the neutral protease dispase are used in the dissociating step. More preferred are those methods which employ digestion in the presence of at least one collagenase from Clostridium histolyticum, and either of the protease activities, dispase and thermolysin. Still more preferred are methods employing digestion with both collagenase and dispase enzyme activities. Also preferred are methods which include digestion with a hyaluronidase activity in addition to collagenase and dispase activities. The skilled artisan will appreciate that many such enzyme treatments are known in the art for isolating cells from various tissue sources. For example, the LIBERASE BLENDZYME (Roche) series of enzyme combinations of collagenase and neutral protease are very useful and may be used in the instant methods. Other sources of enzymes are known, and the skilled artisan may also obtain such enzymes directly from their natural sources. The skilled artisan is also well-equipped to assess new, or additional enzymes or enzyme combinations for their utility in isolating the cells of the disclosure. Preferred enzyme treatments are 0.5, 1, 1.5, or 2 hours long or longer. In other preferred embodiments, the tissue is incubated at 37.degree. C. during the enzyme treatment of the dissociation step. Diluting the digest may also improve yields of cells as cells may be trapped within a viscous digest.

While the use of enzyme activities is presently preferred, it is not required for isolation methods as provided herein. Methods based on mechanical separation alone may be successful in isolating the instant cells from the umbilicus as discussed above.

The cells can be resuspended after the tissue is dissociated into any culture medium as discussed herein above. Cells may be resuspended following a centrifugation step to separate out the cells from tissue or other debris. Resuspension may involve mechanical methods of resuspending, or simply the addition of culture medium to the cells.

Providing the growth conditions allows for a wide range of options as to culture medium, supplements, atmospheric conditions, and relative humidity for the cells. A preferred temperature is 37.degree. C., however, the temperature may range from about 35.degree. C. to 39.degree. C. depending on the other culture conditions and desired use of the cells or culture.

Presently preferred are methods which provide cells which require no exogenous growth factors, except as are available in the supplemental serum provided with the Growth Medium. Also provided herein are methods of deriving umbilical cells capable of expansion in the absence of particular growth factors. The methods are similar to the method above; however they require that the particular growth factors (for which the cells have no requirement) be absent in the culture medium in which the cells are ultimately resuspended and grown in. In this sense, the method is selective for those cells capable of division in the absence of the particular growth factors. Preferred cells in some embodiments are capable of growth and expansion in chemically defined growth media with no serum added. In such cases, the cells may require certain growth factors, which can be added to the medium to support and sustain the cells. Presently preferred factors to be added for growth on serum-free media include one or more of FGF, EGF, IGF, and PDGF. In more preferred embodiments, two, three or all four of the factors are add to serum free or chemically defined media. In other embodiments, LIF is added to serum-free medium to support or improve growth of the cells.

Also provided are methods wherein the cells can expand in the presence of from about 5% to about 20% oxygen in their atmosphere. Methods to obtain cells that require L-valine require that cells be cultured in the presence of L-valine. After a cell is obtained, its need for L-valine can be tested and confirmed by growing on D-valine containing medium that lacks the L-isomer.

Methods are provided wherein the cells can undergo at least 25, 30, 35, or doublings prior to reaching a senescent state. Methods for deriving cells capable of doubling to reach 10.sup.14 cells or more are provided. Preferred are those methods which derive cells that can double sufficiently to produce at least about 10.sup.14, 10.sup.15, 10.sup.16, or or more cells when seeded at from about 10.sup.3 to about 10.sup.6 cells/cm.sup.2 in culture. Preferably these cell numbers are produced within 80, 70, or 60 days or less. In one embodiment, cord tissue mesenchymal stem cells are isolated and expanded, and possess one or more markers selected from a group comprising of CD10, CD13, CD44, CD73, CD90, CD141, PDGFr-alpha, or HLA-A,B,C. In addition, the cells do not produce one or more of CD31, CD34, CD45, CD117, CD141, or HLA-DR,DP, DQ.

In one embodiment, bone marrow MSC lots are generated, means of generating BM MSC are known in the literature and examples are incorporated by reference.

In one embodiment BM-MSC are generated as follows:
1. 500 mL Isolation Buffer is prepared (PBS+2% FBS+2 mM EDTA) using sterile components or filtering Isolation Buffer through a 0.2 micron filter. Once made, the Isolation Buffer was stored at 2-8.degree. C.
2. The total number of nucleated cells in the BM sample is counted by taking 10.mu.L BM and diluting it 1/50-1/100 with 3% Acetic Acid with Methylene Blue (STEMCELL Catalog #07060). Cells are counted using a hemacytometer.
3. 50 mL Isolation Buffer is warmed to room temperature for 20 minutes prior to use and bone marrow was diluted 5/14 final dilution with room temperature Isolation Buffer (e.g. 25 mL BM was diluted with 45 mL Isolation Buffer for a total volume of mL).
4. In three 50 mL conical tubes (BD Catalog #352070), 17 mL Ficoll-Paque™. PLUS (Catalog #07907/07957) is pipetted into each tube. About 23 mL of the diluted BM from step 3 was carefully layered on top of the Ficoll-Paque.™. PLUS in each tube.
5. The tubes are centrifuged at room temperature (15-25.degree. C.) for 30 minutes at 300.times.g in a bench top centrifuge with the brake off.
6. The upper plasma layer is removed and discarded without disturbing the plasma:Ficoll-Paque.™. PLUS interface. The mononuclear cells located at the interface layer are carefully removed and placed in a new 50 mL conical tube. Mononuclear cells are resuspended with 40 mL cold (2-8.degree. C.) Isolation Buffer and mixed gently by pipetting.
7. Cells were centrifuged at 300.times.g for 10 minutes at room temperature in a bench top centrifuge with the brake on. The supernatant is removed and the cell pellet resuspended in 1-2 mL cold Isolation Buffer.
8. Cells were diluted 1/50 in 3% Acetic Acid with Methylene Blue and the total number of nucleated cells counted using a hemacytometer.
9. Cells are diluted in Complete Human MesenCult®-Proliferation medium (STEMCELL catalog #05411) at a final concentration of 1.times.10.sup.6 cells/mL.
BM-derived cells were ready for expansion and CFU-F assays in the presence of GW2580, which can then be used for specific applications.

In one embodiment, MSC are generated according to protocols previously utilized for treatment of patients utilizing bone marrow derived MSC. Specifically, bone marrow is aspirated (10-30 ml) under local anesthesia (with or without sedation) from the posterior iliac crest, collected into sodium heparin containing tubes and transferred to a Good Manufacturing Practices (GMP) clean room. Bone marrow cells are washed with a washing solution such as Dulbecco's phosphate-buffered saline (DPBS), RPMI, or PBS supplemented with autologous patient plasma and layered on to 25 ml of Percoll (1.073 g/ml) at a concentration of approximately 1-2′10⁷ cells/ml. Subsequently the cells are centrifuged at 900 g for approximately 30 min or a time period sufficient to achieve separation of mononuclear cells from debris and erythrocytes. Said cells are then washed with PBS and plated at a density of approximately 1′10⁶ cells per ml in 175 cm 2 tissue culture flasks in DMEM with 10% FCS with flasks subsequently being loaded with a minimum of 30 million bone marrow mononuclear cells. The MSCs are allowed to adhere for 72 h followed by media changes every 3-4 days. Adherent cells are removed with 0.05% trypsin-EDTA and replated at a density of 1′10⁶ per 175 cm². Said bone marrow MSC may be administered intravenously, or in a preferred embodiment, intrathecally in a patient suffering radiation associated neurodegenerative manifestations. Although doses may be determined by one of skill in the art, and are dependent on various patient characteristics, intravenous administration may be performed at concentrations ranging from 1-10 million MSC per kilogram, with a preferred dose of approximately 2-5 million cells per kilogram.

Cell cultures are tested for sterility weekly, endotoxin by limulus amebocyte lysate test, and mycoplasma by DNA-fluorochrome stain.

In order to determine the quality of MSC cultures, flow cytometry is performed on all cultures for surface expression of SH-2, SH-3, SH-4 MSC markers and lack of contaminating CD14- and CD-45 positive cells. Cells were detached with 0.05% trypsin-EDTA, washed with DPBS+2% bovine albumin, fixed in 1% paraformaldehyde, blocked in 10% serum, incubated separately with primary SH-2, SH-3 and SH-4 antibodies followed by PE-conjugated anti-mouse IgG(H+L) antibody. Confluent MSC in 175 cm 2 flasks are washed with Tyrode's salt solution, incubated with medium 199 (M199) for 60 min, and detached with trypsin-EDTA (Gibco). Cells from 10 flasks were detached at a time and MSCs were resuspended in 40 ml of M199+1% human serum albumin (HSA; American Red Cross, Washington DC, USA). MSCs harvested from each 10-flask set were stored for up to 4 h at 4° C. and combined at the end of the harvest. A total of 2-10′10⁶ MSC/kg were resuspended in M199+1% HSA and centrifuged at 460 g for 10 min at 20° C. Cell pellets were resuspended in fresh M199+1% HSA media and centrifuged at 460 g for 10 min at 20° C. for three additional times. Total harvest time was 2-4 h based on MSC yield per flask and the target dose. Harvested MSC were cryopreserved in Cryocyte (Baxter, Deerfield, IL, USA) freezing bags using a rate controlled freezer at a final concentration of 10% DMSO (Research Industries, Salt Lake City, UT, USA) and 5% HSA. On the day of infusion cryopreserved units were thawed at the bedside in a 37° C. water bath and transferred into 60 ml syringes within 5 min and infused intravenously into patients over 10-15 min. Patients are premedicated with 325-650 mg acetaminophen and 12.5-25 mg of diphenhydramine orally. Blood pressure, pulse, respiratory rate, temperature and oxygen saturation are monitored at the time of infusion and every 15 min thereafter for 3 h followed by every 2 h for 6 h.

In one embodiment of the disclosure MSC to be used for induction of post mortem organ regeneration are transfected with anti-apoptotic proteins to enhance in vivo longevity. The present disclosure includes a method of using MSC that have been cultured under conditions to express increased amounts of at least one anti-apoptotic protein as a therapy to inhibit or prevent apoptosis. In one embodiment, the MSC which are used as a therapy to inhibit or prevent apoptosis have been contacted with an apoptotic cell. The disclosure is based on the discovery that MSC that have been contacted with an apoptotic cell express high levels of anti-apoptotic molecules. In some instances, the MSC that have been contacted with an apoptotic cell secrete high levels of at least one anti-apoptotic protein, including but not limited to, STC-1, BCL-2, XIAP, Survivin, and Bc1-2XL. Methods of transfecting antiapoptotic genes into MSC have been previously described which can be applied to the current disclosure, said antiapoptotic genes that can be utilized for practice of the disclosure, in a nonlimiting way, include GATA-4 [8], FGF-2 [9], bcl-2 [10, 11], and HO-1 [12]. Based upon the disclosure provided herein, MSC can be obtained from any source. The MSC may be autologous with respect to the recipient (obtained from the same host) or allogeneic with respect to the recipient. In addition, the MSC may be xenogeneic to the recipient (obtained from an animal of a different species). In one embodiment of the disclosure MSC are pretreated with agents to induce expression of antiapoptotic genes, one example is pretreatment with exendin-4 as previously described [13]. In a further non-limiting embodiment, MSC used in the present disclosure can be isolated, from the bone marrow of any species of mammal, including but not limited to, human, mouse, rat, ape, gibbon, bovine. In a non-limiting embodiment, the MSC are isolated from a human, a mouse, or a rat. In another non-limiting embodiment, the MSC are isolated from a human.

Based upon the present disclosure, MSC can be isolated and expanded in culture in vitro to obtain sufficient numbers of cells for use in the methods described herein provided that the MSC are cultured in a manner that promotes contact with a tumor endothelial cell. For example, MSC can be isolated from human bone marrow and cultured in complete medium (DMEM low glucose containing 4 mM L-glutamine, 10% FBS, and 1% penicillin/streptomycin) in hanging drops or on non-adherent dishes. The disclosure, however, should in no way be construed to be limited to any one method of isolating and/or to any culturing medium. Rather, any method of isolating and any culturing medium should be construed to be included in the present disclosure provided that the MSC are cultured in a manner that provides MSC to express increased amounts of at least one anti-apoptotic protein. Culture conditions for growth of clinical grade MSC have been described in the literature and are incorporated by reference [14-47].

Cell cultures are tested for sterility weekly, endotoxin by limulus amebocyte lysate test, and mycoplasma by DNA-fluorochrome stain.

For the practice of the in disclosure MSC may be purified from various cellular sources such as bone marrow cells [48-54], umbilical cord tissue [55-57], peripheral blood [58-60], amniotic membrane [61], amniotic fluid, mobilized peripheral blood [62], adipose tissue [63, 64], endometrium and other tissues. When tissue sources of MSC are used said tissue isolates from which the MSC are isolated comprise a mixed populations of cells. MSC with endometrial stimulating activity constitute a very small percentage in these initial populations. They must be purified away from the other cells before they can be expanded in culture sufficiently to obtain enough cells for therapeutic applications.

There are several causes of brain death. In order for the practitioner of the disclosure to be assisted, we describe some of the aspects related to brain death. The process of brain death may be started by an initiation of brain cell loss of viability (usually but not exclusively through necrosis) which may be due to different causes. Such necrosis may result in increased osmotic pressure in the brain, resulting in water absorption via the blood-brain barrier. Since the skull cannot expand, the intracranial pressure rises considerably. This is one of the reasons why in certain cases of brain injury, decompressive craniotomy is performed. During cases of brain injury or necrotic cell death, when the intracranial pressure exceeds the systolic blood pressure, the brain starts to become ischemic. This is due to the fact that entry of blood into the brain is compromised. As a response to the lack of brain perfusion, the starts to signals to stimulate an enhanced heart rate and blood flow. Additionally, the body response by increasing the systemic vascular resistance. Another aspect of the response to increased intracranial pressure is that, the adrenal gland induces the Cushing reflex, which is an increase in the level of circulating adrenalin (epinephrine) and nor-adrenaline (nor-epinephrine). As part of this cascade, the heart rate may increase by several hundred percent, to a maximum heart rate. The blood pressure may increase to above 200 mmHg. This massive reaction is also called the “catecholamine storm” or “sympathetic autonomous storm”. The adrenaline and/or nor-adrenaline levels may increase by 70 times, as described in more detail below. If this increase of systolic blood pressure is insufficient for delivering blood to the brain, the brain will maintain its ischemic state. However, the brain cannot sustain more than about 10 minutes without blood supply. If the intracranial pressure, for example due to the increased osmotic pressure, rises to more than about 300 mmHg, the brain cannot withstand such high pressures but disintegrates. The end result will be a progressive brain swelling, and herniation of the hippocampal gyri with lateral pressure of the brainstem, with eventual loss of brainstem function and loss of spontaneous respiration. This may results in herniation of the brain stem through the foramen magnum.

For the process of organ collection, there are various definitions of brain death, one definition is irreversible loss of function of the entire brain including the brainstem. There are several indicia of brain death, which are of less interest for the present embodiments. However, after brain death, there is no cerebral blood circulation and no spontaneous respiration. The body temperature should be above 33.degree. C. and there should be no drug intoxication. It is known that subsequent to brain death, the brain, including the brain stem, cannot retain its function, because it is permanently damaged. Associated with the process of brain death, the stimulation of the “catecholamine storm”, the levels of adrenaline and nor-adrenaline may rise considerably.

It is also established that after brain death, the hypothalamic-pituitary-adrenal axis is disrupted. However, necrosis is followed by a release of cytokines, especially IL-6, which stimulates the adrenal gland to produce adrenaline and nor-adrenaline. Eventually, the production of these inotropics will be reduced and after some 60 minutes, the levels of adrenaline and nor-adrenaline will be lower than normal. This will result in vasoplegia by loss of the sympathetic vasotonus. The pituitary gland also produces antidiuretic hormone (ADH), or vasopressin, which acts on the kidneys in order to control the water resorption. ADH has a short half-life-time of about 15 minutes and a shortage of ADH will occur after some 60 minutes (with large individual variations). Depletion of ADH may result in diabetes insipidus, resulting in production of large quantities of urine in the order of several liters per hour. Unless replacement of fluid takes place, diabetes insipidus will result in hypovolemia, a further reduction of blood pressure and eventual loss of circulation, resulting in ischemic damage of all organs. Moreover, the pituitary gland produces adrenocorticotropic hormone (ACTH), which stimulates secretion of glucocorticoids, which stimulates the synthesis of adrenaline and nor-adrenaline. In addition, the pituitary gland produces thyroid-stimulating hormone (TSH), which stimulates the thyroid gland to secrete the hormones thyroxin (T4) and triiodotyronine (T3). Depletion of T4 and T3 may result in a change from aerobic to anaerobic metabolism in for example the heart, which may result in increased lactate and pyruvate levels.

In one embodiment of the disclosure, the administration regenerative cells is performed in order to repair donor brain structures. It is known that because the pituitary gland is dependent on the hypothalamus, the operation of the pituitary gland is reduced or ceases, resulting in decreased circulating levels of for example T3, T4, ADH, ACTH, cortisol and insulin. This results in impaired aerobic metabolism, increased anaerobic metabolism, depletion of high-energy phosphates and increased lactate production. Side effects of high levels of catecholamines are tachycardia, atrial and ventricular arrhythmias as well as conduction abnormalities. Pulmonary edema may result after high levels of catecholamines, especially adrenaline. Because of vasoconstriction caused by catecholamines, the organs may lose perfusion. Hypothalamus controls body temperature, and the failure of the hypothalamus may result in hypothermia. There are a number of different strategies suggested in the literature for maintaining organs after brain death. The fact that it is possible with prolonged somatic support has been reported for a pregnant woman with brain death. By full ventilatory and nutritional support, vasoactive drugs, maintenance of normothermia, hormone replacement and other supportive measures, the fetus could be born several weeks after brain death of the mother, thereby improving the survival prognosis for the fetus. In maintaining donor viability it is necessary to alter various processes that are deranged as a result of the altered hemostatic processes. One such derangement is the drop in ADH. Depletion of ADH sooner or later results in diabetes insipidus with high urine volumes leading to hypovolemia. This has been counteracted by infusion of large volumes of colloidal or crystalloid fluid, such as Ringer's solution. Another approach is to add a vasopressor agent, such as arginine vasopressin, desmopressin, DDAVP or Minirin. To maintain viability of the brain dead patient, the decrease of thyroid hormone level should also be addressed in order not to aggravate metabolism. Thus, addition of T4 and/or T3 may be appropriate. The reduction in ACTH and cortisol may be addressed by giving methylprednisolone, or a similar agent. In order to maintain proper perfusion of the organs, especially the kidney, it is considered that a mean arterial pressure MAP of at least 60 mmHg should be upheld, see for example the above-mentioned review article. This may be done by adding catecholamines, such as nor-adrenaline and/or adrenaline. However, there is evidence that addition of adrenaline and/or nor-adrenaline may aggravate the conditions for some organs, and there is a tendency in the art to avoid the addition of catecholamines. Traditionally, dopamine has been the inotrope of choice in doses titrated to ensure cardiac output and vasoconstriction to ensure perfusion pressure gradients to the myocardium and the renal circulation. Catecholamines have a half-life of approximately a few minutes when circulating in blood. Normal secretion in the adrenal medulla of adrenaline is 0.2.mu.g/kg/min and of nor-adrenaline 0.05.mu.g/kg/min. It is reported in the literature that administration of nor-adrenaline has been associated with myocardial damage and initial nonfunctioning after cardiac transplantation. It is hypothesized that the “catecholamine storm” after brain death may cause myocardial ischemia or rapid desensitization of the beta-adrenergic signaling pathway. Administration of further nor-adrenaline after brain death may further desensitize the myocardial beta-adrenergic signaling. Another possible explanation might be that, under massive catecholamine release, the uptake and inactivation metabolization systems may be saturated, resulting in a down-regulation of beta adrenergic cardiac receptors (BAR), i.e. a reduction of BAR density, which may be dose dependent. The recovery potential of BAR remains unknown, but may have an impact on organ function.

In addition, catecholamines may sulfoconjugate, which is regarded as an inactivation process by which the organism “pools” free plasma catecholamines into inactivated derivates, which subsequently are deconjutaged and released. Thus, there is evidence that high levels of catecholamines may impair the alpha- and/or beta-receptors potency. In addition, the elimination system may be saturated, which may finally result in poor graft outcome. A fundamental idea of the present embodiments is to replace at least some of the substances and/or hormones that are no longer excreted, or are excreted in substantially lower levels, by the brain dead body compared to a living body. The focus is to maintain hemodynamic stability by cardiovascular support because it may maintain all of the donor organs in the best possible condition. The inventor has found that adrenaline and nor-adrenaline are two substances that would be beneficial to add, but the addition of either of the substances is controversial and may result in undesired side effects as mentioned above. Although the exact mechanism is unknown today, it is believed that a high level of catecholamines, such as under the “catecholamine storm” will cause a depletion of the stores of catecholamine normally found in the nerve terminals and adrenal medulla. In addition, the vascular tonus is lost, because the nerve terminals receive no signals from the brain. Nor-adrenaline is normally produced in the pre-synaptic nerve terminal from tyrosine, which is an amino acid present all over the body in large quantities.

The body is provided with proper respiratory ventilation to keep the partial pressures of oxygen and carbon dioxide at suitable levels. Normally the brain dead body has no spontaneous respiration, which means that active ventilation is required. Such ventilation may take place in any manner previously known, for example by a respirator, by external compression of thorax, by manual or mechanical means. The body is also provided with an infusion solution for maintaining fluid balance. The kidneys produce urine at a desired output level of at least 1.0 ml/kg/hour. Thus, a fluid, such as Kreb's Ringer's solution, is infused at a rate of about 1 to 5 ml/kg/hour to compensate for kidney output, sweat and fluid losses during respiration. The patient may still further be treated with compositions that may further contain additional components such as cortisone, thyroxin (T4), insulin, triiodotyronine (T3), a vasopressor agent, such as arginine vasopressin, desmopressin or Minirin, and methylprednisolone (cortisone). In order to avoid diabetes insipidus, it may be proper to add desmopressin already as early as possible, for example a bolus at the start of the intervention and then a normal continuous dose as produced by the body. Desmopressin may be titrated in dependence of the urine output, in order to maintain the goal of for example 1.0 ml/kg/hour. Since the urine output immediately after the catecholamine storm is very small or even non-existent, it may be required to add a Diuretic agent, such as Furosemide (LASIX) in order to start urine production. In treatment of patients may be performed using means disclosed in the art, such as in patent application one example the patent application #20110270215 which describes composition comprises the NET inhibitor, such as cocaine, and in addition adrenaline, nor-adrenaline, cortisone, thyroxin, triiodotyronine, and desmopressin. The ratio between the NET inhibitor:nor-adrenaline may be about 1:1. In some embodiments, the adrenaline and/or nor-adrenaline may be partly or entirely replaced by an equivalent substance. For example, phenylephrine is an alpha-1-agonist and may replace nor-adrenaline. It seems that phenylephrine is about 5 times less potent as nor-adrenaline. Dopamine may be added in quantities less than about 0.01 mg/kg/min. The embodiments also relate to an infusion solution comprising the composition as defined above dissolved in a pharmaceutical acceptable medium. Examples of acceptable mediums are physiological sodium chloride solution, Hartmann's solution and Ringer's (acetate) solution. Since the added volume is very small, in the range of 1.7 ml/hour (=0.04 ml/kg/hour), the ingredients may be dissolved in sterile, non-ionic water, i.e. pure H.sub.20. The final amounts of the different components, which may be present in the infusion solution of a volume of 50 ml, are about 0.1 to about 10 mg of nor-adrenaline, for example 1 mg, 0.1 to about 10 mg of adrenaline, such as 1 mg, 0.1 to about 10 mg of the NET inhibitor, such as 1 mg. The other components, which may be present, may be in an amount of about 0.05 to about 3 mg of triiodotyronine, T3, about 100 to about 1000 mg hydrocortisone, insulin and desmopressin.

In one embodiment the disclosure provides means of inducing post-mortem regeneration by administration of endothelial progenitor cells. These cells are capable of differentiating into endothelial cells which can induce recovery of damaged endothelium. The endothelium plays a critical role in the function of organs and provides means of inducing in vivo regeneration. The examples of the importance of endothelial health can be seen in various biological systems. For example, dilatation response serves as a means of quantifying one aspect of endothelial health [65, 66]. This assay has been used to show endothelial dysfunction in conditions such as healthy aging [67-69], as well as various diverse inflammatory states including renal failure [70], rheumatoid arthritis [71], Crohn's Disease [72], diabetes [73], heart failure [74], and Alzheimer's [75]. Although it is not clear whether reduction in FMD score is causative or an effect of other properties of endothelial dysfunction, it has been associated with: a) increased tendency towards thrombosis, in part by increased vWF levels [76], b) abnormal responses to injury, such as neointimal proliferation and subsequent atherosclerosis [77], and c) increased proclivity towards inflammation by basal upregulation of leukocyte adhesion molecules [78].

As part of age and disease associated endothelial dysfunction is the reduced ability of the host to generate new blood vessel [79]. This is believed to be due, at least in part, to reduction of ischemia inducible elements such as the HIF-1 alpha transcription factor which through induction of SDF-1 and VEGF secretion play a critical role in ability of endothelium to migrate and form new capillaries in ischemic tissues [80, 81]. Accordingly, if one were to understand the causes of endothelial dysfunction and develop methods of inhibiting these causes or stimulating regeneration of the endothelium, then progression of many diseases, as well as possible increase in healthy longevity may be achieved.

The endothelium plays several functions essential for life, including: a) acting as an anticoagulated barrier between the blood stream and interior of the blood vessels; b) allowing for selective transmigration of cells into and out of the blood stream; c) regulating blood flow through controlling smooth muscle contraction/relaxation; and d) participating in tissue remodeling [82]. A key hallmark of the aging process and perhaps one of the causative factors of health decline associated with aging appears to be loss of endothelial function. Whether as a result of oxidative stress, inflammatory stress, or senescence, deficiencies in the ability of the endothelium to respond to physiological cues can alter the ability to think [83], procreate [84], see [85], and breathe [86]. Specifically, minute alterations in the ability of endothelium to respond to neurotransmitter induced nitric oxide causes profound inability to perform even simple mental functions [87, 88]. Small increases in angiogenesis in the retina as a result of injury or glucose are associated with wet macular degeneration blindness [89]. Atherosclerosis of the penile vasculature is a major cause of erectile dysfunction [90]. The pulmonary endothelium's sensitivity to insult can cause hypertension and associated progression to decreased oxygen delivery [91].

In one embodiment of the disclosure endothelial progenitor cells are administered to the post-mortem patient and FMD is utilized as a assay system to determine the amount of regeneration achieved by infusion of regenerative cells.

A key component of endothelial turnover appears to be the existence of circulating endothelial progenitor (EPC) cells that appear to be involved in repair and angiogenesis of ischemic tissues. An early study in 1963 hinted at the existence of such circulating EPC after observations of endothelial-like cells, that were non-thrombogenic and morphologically appeared similar to endothelium, were observed covering a Dacron graft that was tethered to the thoracic artery of a pig [92]. The molecular characterization of the EPC is usually credited to a 1997 paper by Asahara et al. in which human bone marrow derived VEGR-2 positive, CD34 positive monocyte-like cells were described as having ability to differentiate into endothelial cells in vitro and in vivo based on expression of CD31, eNOS, and E-selectin [93]. These studies were expanded into hindlimb ischemia in mouse and rabbit models in which increased circulation of EPC in response to ischemic insult was observed [94]. Furthermore, these studies demonstrated that cytokine-induced augmentation of EPC mobilization elicited a therapeutic angiogenic response. Using irradiated chimeric systems, it was demonstrated that ischemia-mobilized EPC derive from the bone marrow, and that these cells participate both in sprouting of pre-existing blood vessels as well as the initiation of de novo blood vessel production [95]. Subsequent to the initial phenotypic characterization by Asahara et al [93], more detailed descriptions of the human EPC were reported. For example, CD34 cells expressing the markers VEGF-receptor 2, CD133, and CXCR-4 receptor, with migrational ability to VEGF and SDF-1 has been a more refined EPC definition [96]. However there is still some controversy as to the precise phenotype of the EPC, since the term implies only ability to differentiate into endothelium. For example, both CD34+, VEGFR2+, CD133+, as well as CD34+, VEGFR2+, CD133− have been reported to act as EPC [97]. More recent studies suggest that the subpopulation lacking CD133 and CD45 are precursor EPC [98]. Other phenotypes have been ascribed to cells with EPC activity, one study demonstrated monocyte-like cells that expressing CD14, Mac-1 and the dendritic cell marker CD11c have EPC activity based on uptake of acetylated LDL and binding to the ulex-lectin [99, 100]. While the initial investigations into the biology of EPC focused around acute ischemia, it appears that in chronic conditions circulating EPC may play a role in endothelial turnover. Apolipoprotein E knockout (ApoE KO) mice are genetically predisposed to development of atherosclerosis due to inability to impaired catabolism of triglyceride-rich lipoproteins. When these mice are lethally irradiated and reconstituted with labeled bone marrow stem cells, it was found that areas of the vasculature with high endothelial turnover, which were the areas of elevated levels of sheer stress, had incorporated the majority of new endothelial cells derived from the bone marrow EPC [101]. The possibility that endogenous bone marrow derived EPC possess such a regenerative function was also tested in a therapeutic setting. Atherosclerosis is believed to initiate from endothelial injury with a proliferative neointimal response that leads to formation of plaques. When bone marrow derived EPC are administered subsequent to wire injury, a substantial reduction in neointima formation was observed [102]. The argument can be made that wire injury of an artery does not resemble the physiological conditions associated with plaque development. To address this, Wassmann et al [103], used ApoE KO mice that were fed a high cholesterol diet and observed reduction in endothelial function as assessed by the flow mediated dilation assay. When EPC were administered from wild-type mice restoration of endothelial responsiveness was observed. In the context of aging, Edelman's group performed a series of interesting experiments in which 3 month old syngeneic cardiac grafts were heterotopically implanted into 18 month old recipients. Loss of graft viability, associated with poor neovascularization, was observed subsequent to transplanting, as well as subsequent to administration of 18 month old bone marrow mononuclear cells. In contrast, when 3 month old bone marrow mononuclear cells were implanted, grafts survived. Antibody depletion experiments demonstrated bone marrow derived PDGF-BB was essential in integration of the young heart cells with the old recipient vasculature [104]. These experiments suggest that young EPC or EPC-like cells have ability to integrate and interact with older vasculature. What would be interesting is to determine whether EPC could be “revitalized” ex vivo by culture conditions or transfection with therapeutic genes such as PDGF-BB. Given animal studies suggest EPC are capable of replenishing the vasculature, and defined markers of human EPC exist, it may be possible to contemplate EPC-based therapies. Two overarching therapeutic approaches would involve utilization of exogenous EPC or mobilization of endogenous cells. Before discussing potential therapeutic interventions, we will first examine several clinical conditions in which increasing circulating EPC may play a role in response to injury.

For the purposes of the disclosure, in some embodiments, the disclosure provides the use of anti-inflammatory agents or approaches for reduction of chronic inflammation. Said chronic inflammation is known to reduce EPC numbers. Through the reduction of EPC it is believed that the ability of exogenous stem cells to function is reduced by suppressed numbers. There is need for angiogenesis and tissue remodeling in the context of various chronic inflammatory conditions. However in many situations it is the aberrant reparative processes that actually contribute to the pathology of disease. Examples of this include: the process of neointimal hyperplasia and subsequent plaque formation in response to injury to the vascular wall [105], the process of hepatic fibrosis as opposed to functional regeneration [106], or the post-infarct pathological remodeling of the myocardium which results in progressive heart failure [107]. In all of these situations it appears that not only the lack of regenerative cells, but also the lack of EPC is present. Conceptually, the need for reparative cells to heal the ongoing damage may have been so overwhelming that it leads to exhaustion of EPC numbers and eventual reduction in protective effect. Supporting this concept are observations of lower number of circulating EPC in inflammatory diseases, which may be the result of exhaustion. Additionally, the reduced telomeric length of EPC in patients with coronary artery disease [108], as well as reduction of telomere length in the EPC precursors that are found in the bone marrow [109, 110] suggests that exhaustion in response to long-term demand may be occurring. If the reparatory demands of the injury indeed lead to depletion of EPC progenitors, then administration of progenitors should have therapeutic effects. Several experiments have shown that administration of EPC have beneficial effects in the disease process. For example, EPC administration has been shown to: decrease balloon injury induced neointimal hyperplasia [111], b) suppress carbon tetrachloride induced hepatic fibrosis [112, 113], and inhibit post cardiac infarct remodeling [114]. One caveat of these studies is that definition of EPC was variable, or in some cases a confounding effect of co-administered cells with regenerative potential may be present. However, overall, there does appear to be an indication that EPC play a beneficial role in supporting tissue regeneration. As discussed below, many degenerative conditions, including healthy aging, are associated with a low-grade inflammation. There appears to be a causative link between this inflammation and reduction in EPC function Inflammatory conditions present with features, which although not the rule, appear to have commonalities. For example, increases in inflammatory markers such as C-reactive protein (CRP), erythrocyte sedimentation rate, and cytokines such as TNF-alpha and IL-18 have been described in diverse conditions ranging from organ degenerative conditions such as heart failure [115, 116], kidney failure [117, 118], and liver failure [119, 120] to autoimmune conditions such as rheumatoid arthritis and Crohn's Disease [122], to healthy aging [123, 124]. Other markers of inflammation include products of immune cells such as neopterin, a metabolite that increases systemically with healthy aging [125], and its concentration positively correlates with cognitive deterioration in various age-related conditions such as Alzheimer's [126]. Neopterin is largely secreted by macrophages, which also produce inflammatory mediators such as TNF-alpha, IL-1, and IL-6, all of which are associated with chronic inflammation of aging [127]. Interestingly, these cytokines are known to upregulate CRP, which also is associated with aging [128]. While there is no direct evidence that inflammatory markers actively cause shorted lifespan in humans, strong indirect evidence of their detrimental activities exists. For example, direct injection of recombinant CRP in healthy volunteers induces atherothrombotic endothelial changes, similar to those observed in aging [129]. In vitro administration of CRP to endothelial cells decreases responsiveness to vasoactive factors, resembling the human age-associated condition of endothelial hyporesponsiveness [130]. Another important inflammatory mediator found elevated in numerous degenerative conditions is the cytokine TNF-alpha. Made by numerous cells, but primarily macrophages, TNF-alpha is known to inhibit proliferation of repair cells in the body, such as oligodendrocytes in the brain [131], and suppress activity of endogenous stem cell pools [132, 133]. TNF-alpha decreases EPC viability, an effect that can be overcome, at least in part by antioxidant treatment [134]. Administration of TNF-alpha blocking agents has been demonstrated to restore both circulating EPC, as well as endothelial function in patients with inflammatory diseases such as rheumatoid arthritis [71, 135, 136],

It appears that numerous degenerative conditions are associated with production of inflammatory mediators, which directly suppress EPC production or activity. This may be one of the reasons for findings of reduced EPC and FMD indices in patients with diverse inflammatory conditions. In addition to the direct effects, the increased demand for de novo EPC production in inflammatory conditions would theoretically lead to exhaustion of EPC precursors cells by virtue of telomere shortening.

Means of decreasing inflammation are known in the art. In some embodiments, administration of anti-inflammatory agents is performed. In some embodiments an antiinflammatory agent is selected from a group comprising of: BLC, Eotaxin-1, Eotaxin-2, G-CSF, GM-CSF, 1-309, ICAM-1, IFN-gamma, IL-1 alpha, IL-1 beta, IL-1 ra, IL-2, IL-4, IL-5, IL-6, IL-6 sR, IL-7, IL-8, IL-10, IL-11, IL-12 p40, IL-12 p′70, IL-13, IL-15, IL-16, IL-17, MCP-1, M-CSF, MIG, MIP-1 alpha, MIP-1 beta, MIP-1 delta, PDGF-BB, RANTES, TIMP-1, TIMP-2, TNF alpha, TNF beta, sTNFRI, sTNFRIIAR, BDNF, bFGF, BMP-4, BMP-5, BMP-7, b-NGF, EGF, EGFR, EG-VEGF, FGF-4, FGF-7, GDF-15, GDNF, Growth Hormone, HB-EGF, HGF, IGFBP-1, IGFBP-2, IGFBP-3, IGFBP-4, IGFBP-6, IGF-1, Insulin, M-CSF R, NGF R, NT-3, NT-4, Osteoprotegerin, PDGF-AA, PIGF, SCF, SCFR, TGFalpha, TGF beta 1, TGF beta 3, VEGF, VEGFR2, VEGFR3, VEGF-D 6Ckine, Axl, BTC, CCL28, CTACK, CXCL16, ENA-78, Eotaxin-3, GCP-2, GRO, HCC-1, HCC-4, IL-9, IL-17F, IL-18 BPa, IL-28A, IL-29, IL-31, IP-10, I-TAC, LIF, Light, Lymphotactin, MCP-2, MCP-3, MCP-4, MDC, MIF, MIP-3 alpha, MIP-3 beta, MPIF-1, MSPalpha, NAP-2, Osteopontin, PARC, PF4, SDF-1 alpha, TARC, TECK, TSLP 4-1BB, ALCAM, B7-1, BCMA, CD14, CD30, CD40 Ligand, CEACAM-1, DR6, Dtk, Endoglin, ErbB3, E-Selectin, Fas, Flt-3L, GITR, HVEM, ICAM-3, IL-1 R4, IL-1 RI, IL-10 Rbeta, IL-17R, IL-2Rgamma, IL-21R, LIMPII, Lipocalin-2, L-Selectin, LYVE-1, MICA, MICB, NRG1-beta1, PDGF Rbeta, PECAM-1, RAGE, TIM-1, TRAIL R3, Trappin-2, uPAR, VCAM-1, XEDARActivin A, AgRP, Angiogenin, Angiopoietin 1, Angiostatin, Catheprin S, CD40, Cripto-1, DAN, DKK-1, E-Cadherin, EpCAM, Fas Ligand, Fcg RIIB/C, Follistatin, Galectin-7, ICAM-2, IL-13 R1, IL-13R2, IL-17B, IL-2 Ra, IL-2 Rb, IL-23, LAP, NrCAM, PAI-1, PDGF-AB, Resistin, SDF-1 beta, sgp130, ShhN, Siglec-5, ST2, TGF beta 2, Tie-2, TPO, TRAIL R4, TREM-1, VEGF-C, VEGFR1Adiponectin, Adipsin, AFP, ANGPTL4, B2M, BCAM, CA125, CA15-3, CEA, CRP, ErbB2, Follistatin, FSH, GRO alpha, beta HCG, IGF-1 sR, IL-1 sRII, IL-3, IL-18 Rb, IL-21, Leptin, MMP-1, MMP-2, MMP-3, MMP-8, MMP-9, MMP-10, MMP-13, NCAM-1, Nidogen-1, NSE, OSM, Procalcitonin, Prolactin, PSA, Siglec-9, TACE, Thyroglobulin, TIMP-4, TSH2B4, ADAM-9, Angiopoietin 2, APRIL, BMP-2, BMP-9, C5a, Cathepsin L, CD200, CD97, Chemerin, DcR3, FABP2, FAP, FGF-19, Galectin-3, HGF R, IFN-gammalpha/beta ?R2, IGF-2, IGF-2 R, IL-1R6, IL-24, IL-33, Kallikrein 14, Legumain, LOX-1, MBL, Neprily sin, Notch-1, NOV, Osteoactivin, PD-1, PGRP-5, Serpin A4, sFRP-3, Thrombomodulin, TLR2, TRAIL R1, Transferrin, WIF-LACE-2, Albumin, AMICA, Angiopoietin 4, BAFF, CA19-9, CD163, Clusterin, CRTAM, CXCL14, Cystatin C, Decorin, Dkk-3, DLL1, Fetuin A, aFGF, FOLR1, Furin, GASP-1, GASP-2, GCSF R, HAI-2, IL-17B R, IL-27, LAG-3, LDL R, Pepsinogen I, RBP4, SOST, Syndecan-1, TALI, TFPI, TSP-1, TRAIL R2, TRANCE, Troponin I, uPA, VE-Cadherin, WISP-1, and RANK.

In some embodiments of the disclosure, EPC are derived from adipose tissues. To date, clinical trials on adipose derived cells have all utilized ex vivo-expanded cells, which share properties with bone marrow derived MSC [137-142]. Preparations of MSC expanded from adipose tissue are equivalent or superior to bone marrow in terms of differentiation ability [143, 144], angiogenesis-stimulating potential [145], and immune modulatory effects [146]. Given the extra processing steps associated with ex vivo expansion of adipose cells, a simpler and perhaps safer procedure would be the use of primary adipose tissue-derived cells for therapy. SVF comprises the mononuclear cells derived from adipose tissue, which are acquired through a simple isolation procedure whereby fat is lipoaspirated and subjected to enzymatic digestion. Currently bench top closed systems for autologous adipose cell therapy, such as Cytori's Celution™ system and Tissue Genesis' TGI 1000™ platform [148], are entering clinical trials.

Although the majority of studies have focused on in vitro expanded adipose derived cells, SVF derived from whole lipoaspirate alleviates the need for extensive processing of the cells, thereby also minimizing the number of steps where contamination could be introduced. An important consideration in clinical scenerios where bulk SVF is utilized is the potential regenerative, angiogenic and immune regulatory contributions of the numerous cellular populations that are present. The mononuclear fraction of adipose tissue, referred to as the stromal vascular fraction (SVF), was originally described as the proliferative component of adipose tissue by Hollenberg et al. in 1968 [149]. The cells comprising SVF morphologically resemble fibroblasts and were demonstrated to differentiate into pre-adipocytes and functional adipose tissue in vitro [150].

Although it was suggested that non-adipose differentiation of SVF may occur under specific conditions [151], the notion of “adipose-derived stem cells” was not widely recognized until a seminal paper in 2001, where Zuk et al demonstrated the SVF contains large numbers of mesenchymal-like stem cells (MSC-like) cells that could be induced to differentiate into adipogenic, chondrogenic, myogenic, and osteogenic lineages [152]. Subsequent to the initial description, the same group reported that in vitro expanded SVF derived cells had surface marker expression similar to bone marrow derived MSC, displaying expression of CD29, CD44, CD71, CD90, CD105/SH2, and SH3 and lacking CD31, CD34, and CD45 expression [153]. MSC are defined as adherent, non-hematopoietic cells expressing the surface markers CD90, CD105, and CD73, while lacking expression of CD14, CD34, and CD45, and having the ability to differentiate into adipocytes, chondrocytes, and osteocytes in vitro after treatment with the appropriate growth factors [154].

Adipose tissue has also been used clinically as a source of regenerative and immune modulatory MSC. Cytori is currently conducting two European clinical trials using autologous, adipose-derived mononuclear cells, of which MSC are believed to be the therapeutic population [155]. The PRECISE trial is a 36-patient safety and feasibility study in Europe evaluating adipose-derived stem and regenerative cells as a treatment for chronic cardiac ischemia. The APOLLO trial is a 48-patient safety and feasibility study in Europe to evaluate adipose-derived regenerative cells as a treatment for heart attacks [156]. Allogeneic uses of adipose derived MSC included treatment of GVHD associated liver failure and steroid refractory GVHD [142, 157], Allogeneic placenta and cord blood-derived MSC have also been used for treatment of heart failure and Buerger's Disease [159], respectively.

From the above-mentioned clinical trials of allogeneic MSC, graft versus host or pathological immunological reactions have not been reported. Additionally, administration of MSC intravenously, intrathecally, and intramuscularly have not been associated with ectopic tissue formation or teratoma. Administration of human MSC has been shown to accelerate hematopoietic reconstitution in animal models [160, 161]. Although the in vivo significance of MSC is still highly debated, one theory is that MSC in the bone marrow provide a suitable environment for hematopoiesis. Accordingly, one of the first clinical uses of MSC has been to accelerate hematopoietic recovery. In a 1995 paper, Lazarus et al. reported the use of autologous, in vitro expanded, “mesenchymal progenitor cells” to treat 15 patients suffering from hematological malignancies in remission. The authors demonstrated feasibility of expanding bone marrow derived by MSC in vitro. They showed that a 10 milliliter bone marrow sample was capable of 16,000-fold growth over a four to seven week in vitro culture period. Cell administration was performed in total doses ranging from 1-50×10 6 cells and was not causative of treatment associated adverse effects [162].

In a subsequent study from the same group in 2000, the use of MSC to accelerate hematopoietic reconstitution was performed in a group of 28 breast cancer patients who received high dose chemotherapy. MSC at concentrations of 1.0-2.2×10 6/kg were administered intravenously. No treatment associated adverse effects where observed, and leukocytic and thrombocytic reconstitution appeared to undergo “rapid recovery” [163]. It is interesting that these initial uses were actually in patients with neoplasia and no overt acceleration of cancer progression was noted. Besides feasibility, these studies were important because they established the technique for ex vivo expansion and readministration. Studies along these lines continued which reaffirmed the feasibility of the approach of “repairing bone marrow stroma” with expanded MSC cells.

In 2005, Lazarus et al treated 46 patients suffering from hematological malignancies with HLA-matched allografts comprising bone marrow and donor-derived expanded MSC. The numbers of MSC administered were 1-5 million/kg. On average, the time to neutrophil reconstitution (as defined by absolute neutrophil count > or =0.500×10⁹/L) and platelet reconstitution (as defined by platelet count > or =20×10⁹/L was 14.0 days (range 11.0-26.0 days) and 20 days (range 15.0-36.0 days). Incidence of acute, Grade II-IV GVHD was 13/46 and chronic was 22/36 patients that survived for at least 90 days. Relapse of malignancy occurred in 11 patients with a median time to progression of 213.5 days (range 14-688 days). The authors concluded that cotransplantation of HLA-identical sibling culture-expanded MSCs with an HLA-identical sibling HSC transplant was feasible and safe, without immediate infusional or late MSC-associated toxicities [164].

These data were of importance since one of the concerns regarding MSC treatment is associated with growth factor production. Leukemic patients have minimally residual disease, which seems to be at least in part controlled by recipient immune function [165, 166]. The demonstration that the recipient did not have an overtly higher incidence of relapse suggests that MSC do not endow a preferential advantage to leukemic cells. This is interesting given that MSC are generally considered immune suppressive cells [167, 168]. In addition to its stem/progenitor cell content, the SVF is known to contain monocytes/macrophages. Although pluripotency of monocytic populations have previously been described [169, 170], we will focus our discussion to immunological properties, specifically, the apparent anti-inflammatory/angiogenic activities of these cells.

Initial experiments suggested that macrophage content of adipose tissue was associated with the chronic low-grade inflammation found in obese patients. This was suggested by co-culture experiments in which adipocytes were capable of inducing TNF-alpha secretion from macrophage cell lines in vitro [171]. Clinical studies demonstrated that adipocytes also directly release a constitutive amount of TNF-alpha and leptin, which are capable of inducing macrophage secretion of inflammatory mediators [172]. Interestingly, it appears from several studies in mice and humans that when monocytes/macrophages are isolated from adipose tissue, they exhibited some phenotype markers of M2 macrophages however the cells also had higher basal and induced levels of the pro-inflammatory mediators, TNF-alpha, IL-6, IL-1, MCP-1, and MIP-1 alpha, compared to levels induced by the pro-inflammatory M1 macrophages [173-175].

If indeed these adipose derived macrophages have an “M2” phenotype, they may be similar to M2 cells observed in conditions of immune suppression such as in tumors [176], post-sepsis compensatory anti-inflammatory syndrome [177, 178], or pregnancy associated decidual macrophages [179]. A recent paper suggested that it is the M2 component of SVF that is associated with enhanced survival of fat grafts that are supplemented with SVF [180]. It is estimated that the monocytic/macrophage compartment of the SVF is approximately 10% based on CD14 expression [181].

Interestingly, administrations of ex vivo generated M2 macrophages have been demonstrated to inhibit kidney injury in an adriamycin-induced model [182]. In the context of multiple sclerosis, alternatively activated, M2-like microglial cells are believed to inhibit progression in the EAE model [183]. Thus, the potential M2 phenotype of adipose derived macrophages may be a mechanism of therapeutic effect of SVF cells when isolated from primary sources and not expanded. It has been reported by us and others, that activation of T cells in the absence of costimulatory signals leads to generation of immune suppressive CD4+ CD25+ T regulatory (Treg) cells [184, 185]. Thus, local activation of immunity in adipose tissue would theoretically be associated with reduced costimulatory molecule expression by the M2 macrophages, which may predispose to Treg generation.

Conversely, it is known that Tregs are involved in maintaining macrophages in the M2 phenotype [186]. Supporting the possibility of Treg in adipose tissue also comes from the high concentration of local MSC which are known to secrete TGF-beta and IL-10[188], both involved in Treg generation [189]. Indeed, numerous studies have demonstrated the ability of MSC to induce Treg cells [188, 190-192]. Over the past two decades the endothelium has received significant attention as a dynamic surface cell that acts as an adaptable, anti-coagulated barrier between the blood stream and interior of the blood vessel. This allows for selective transmigration of cells in and out of the blood stream, regulates blood flow through controlling smooth muscle contraction, and participates in tissue remodeling and angiogenesis [82-86]. Endothelial cells are believed to originate from a primitive stem cell, the hemangioblast, which is capable of giving rise to both hematopoietic and endothelial cells [193]. During adulthood, the endothelium is continually self-renewed by a population of bone marrow-derived cells termed endothelial progenitor cells (EPC). This progenitor population has previously been characterized as expressing the CD34 HSC marker as well as VEGF-receptor 2 and AC133 [96]. These cells have been demonstrated using in vivo chimeric models to repair damaged blood vessels in non-diseased as well as in pathological settings [102, 103].

The invention describes a self-regulating device possessing ability to induce regenerative changes systemically in a brain dead patient. The basic concept of the device is to create negative feedback proportional to the exact degree of associated inflammatory stimuli, in some cases, said brain death associated stimuli being inflammation. More precisely, several inflammatory mediators known as cytokines (protein hormones that induce, modulate, and augment inflammation) are associated with various aspects of brain death. Without being bound to theory, the device described represents and exogenous bioreactor in which regenerative cells are in contact with circulating factors derived from a brain dead individual and produce regenerative factors in response. In some embodiments of the invention, brain death-associated factors are inflammatory mediators. In some embodiments of the invention, regenerative factors are agents such as GDF-11, exosomes, or other agents associated with regeneration such as BDNF, EGF, hCG, VEGF, and IGF-1.

In one embodiment of the device, the device produces or releases one or more units of regenerative factors for every one or more unit of a given degenerative factors, such as an inflammatory cytokine. The device described comprises of a biohybrid device, in which regenerative cells are housed in a bioreactor or matrix. Said regenerative cells housed in said device serve to 1) sense the levels of a given age-associated factor(s); 2) produce the appropriate levels of the appropriate counteracting regenerative factor(s); and 3) possibly also release diagnostic markers that would serve to either delineate the degree of degenerative factor(s) produced by the patient.

In one embodiment, a bioreactor is therefore provided, for example, comprising a compartment comprising regenerative cells. The bioreactor comprising a selectively permeable membrane in contact with the cells. The selectively permeable membrane can be a selectively permeable hollow fiber. Alternately, the compartment comprising the cells can comprise a vessel having a selectively permeable wall. The vessel may comprise a plurality of selectively permeable hollow fibers passing through the compartment through which one or both of a gas and a fluid comprising nutrients for the cells can be passed. In another embodiment, the compartment comprising the cells comprises a plurality of selectively permeable hollow fibers passing through the compartment in which the plurality of hollow fibers are fluidly connected to a plasma or blood circulation system in which blood or plasma from the patient can be circulated through the hollow fibers and into a patient.

In further embodiments, the device contains a compartment comprising the cells and comprises a plurality of selectively permeable hollow fibers passing through the compartment in which the plurality of hollow fibers are fluidly connected to a plasma or blood circulation system in which blood or plasma from the patient is circulated through the hollow fibers and into the patient and/or an isolated organ of the patient. In another embodiment, the compartment comprising the cells has at least one wall that is the selectively permeable membrane, in which the first side of the membrane is placed in contact with a wound on the patient or a bodily fluid in situ in the patient. In that embodiment, optionally, the compartment comprises a plurality of selectively permeable hollow fibers passing through the compartment through which one or both of a gas and a fluid comprising nutrients for the cells is passed.

Regenerative cells for use with said device may be any cell that is effective in its use in the bioreactor, and may be xenogeneic, syngeneic, allogeneic, or autologous cells to a patient treated by use of the bioreactor.

In one embodiment of the invention, said regenerative cells are amniotic fluid stem cells. Said amniotic fluid stem cells The amniotic fluid-derived stem cells described in this invention are capable of self-renewal in tissue culture, maintain euploidy for >1 year in culture, share markers with human ES cells, and are capable of differentiating into all three germ layers of the developing embryo, Endoderm, Mesoderm and Ectoderm. In a preferred embodiment the regenerative amniotic fluid cells are found in the amnion harvested during the second trimester of human pregnancies. It is known that amniotic fluid contains multiple morphologically-distinguishable cell types, the majority of the cells are prone to senescence and are lost from cultures. In one embodiment, fibronectin coated plates and culture conditions described in U.S. Pat. No. 7,569,385 are used to grow cells from amniotic fluid harvests from normal 16-18 week pregnancies. The cells of the invention are of fetal origin, and have a normal diploid karyotype. Growth of the amniotic fluid stem cells as described in the invention for use in neurological ischemic conditions results in cells that are multipotent, as several main cell types have been derived from them. As used herein, the term “multipotent” refers to the ability of amniotic fluid regenerative cells to differentiate into several main cell types. The MAFSC cells may also be propagated under specific conditions to become “pluripotent.” The term “pluripotent stem cells” describes stem cells that are capable of differentiating into any type of body cell, when cultured under conditions that give rise to the particular cell type. The Amniotic fluid regenerative cells are preferably isolated from humans. However, the Amniotic fluid regenerative cells may be isolated in a similar manner from other species. Examples of species that may be used to derive the Amniotic fluid regenerative cells include but are not limited to mammals, humans, primates, dogs, cats, goats, elephants, endangered species, cattle, horses, pigs, mice, rabbits, and the like.

The amniotic fluid-derived cells and MAFSC can be recognized by their specific cell surface proteins or by the presence of specific cellular proteins. Typically, specific cell types have specific cell surface proteins. These surface proteins can be used as “markers” to determine or confirm specific cell types. Typically, these surface markers can be visualized using antibody-based technology or other detection methods.

The surface markers of the isolated MAFSC cells derived from independently-harvested amniotic fluid samples were tested for a range of cell surface and other markers, using monoclonal antibodies and FACS analysis. These cells can be characterized by the following cell surface markers: SSEA3, SSEA4, Tra-1-60, Tra-1-81, Tra-2-54. The MAFSC cells can be distinguished from mouse ES cells in that the MAFSC cells do not express the cell surface marker SSEA1. Additionally, MAFSC express the stem cell transcription factor Oct-4. The MAFSC cells can be recognized by the presence of at least one, or at least two, or at least three, or at least four, or at least five, or at least six, or all of the following cellular markers SSEA3, SSEA4, Tra-1-60, Tra-1-81, Tra-2-54 and Oct-4.

The MAFSC cultures express very little or no SSEA-1 marker. In addition to the embryo stem cell markers SSEA3, SSEA4, Tra-1-60, Tra-1-81, Tra2-54, Oct-4 the amniotic fluid regenerative cells also expressed high levels of the cell surface antigens that are normally found on human mesenchymal stem cells, but not normally on human embryo stem cells. This set of markers includes CD13 (99.6%) aminopeptidase N, CD44 (99.7%) hyaluronic acid-binding receptor, CD49b (99.8%) collagen/laminin-binding integrin alpha2, and CD105 (97%) endoglin. The presence of both the embryonic stem cell markers and the hMSC markers on the MAFSC cell cultures indicates that amniotic fluid-derived MAFSC cells, grown and propagated as described here, represent a novel class of human stem cells that combined the characteristics of hES cells and of hMSC cells.

In some embodiments of the invention, at least about 90%, 94%, 97%, 99%, or 100% of the cells in the culture express CD13. In additional embodiments, at least about 90%, 94%, 97%, 99%, or 100% of the cells in the culture express CD44. In some embodiments of the invention, a range from at least about 90%, 94%, 97%, 99%, 99.5%, or 100% of the cells in the culture express CD49b. In further embodiments of the invention, a range from at least about 90%, 94%, 97%, 99%, 99.5%, or 100% of the cells in the culture express CD105.

In one particular embodiment of the invention, the amniotic fluid regenerative cells are human stem cells that can be propagated for an indefinite period of time in continuous culture in an undifferentiated state. The term “undifferentiated” refers to cells that have not become specialized cell types. A “nutrient medium” is a medium for culturing cells containing nutrients that promote proliferation. The nutrient medium may contain any of the following in an appropriate combination: isotonic saline, buffer, amino acids, antibiotics, serum or serum replacement, and exogenously added factors. The Amniotic fluid regenerative cells may be grown in an undifferentiated state for as long as desired (and optionally stored as described above), and can then be cultured under certain conditions to allow progression to a differentiated state. By “differentiation” is meant the process whereby an unspecialized cell acquires the features of a specialized cell such as a heart, liver, muscle, pancreas or other organ or tissue cell. The Amniotic fluid regenerative cells, when cultured under certain conditions, have the ability to differentiate in a regulated manner into three or more subphenotypes. Once sufficient cellular mass is achieved, cells can be differentiated into endodermal, mesodermal and ectodermal derived tissues in vitro and in vivo. This planned, specialized differentiation from undifferentiated cells towards a specific cell type or tissue type is termed “directed differentiation.” Exemplary cell types that may be prepared from Amniotic fluid regenerative cells using directed differentiation include but are not limited to fat cells, cardiac muscle cells, epithelial cells, liver cells, brain cells, blood cells, neurons, glial cells, pancreatic cells, and the like.

General methods relating to stem cell differentiation techniques that may be useful for differentiating the Amniotic fluid regenerative cells of this invention can be found in general texts such as: Teratocarcinomas and embryonic stem cells: A practical approach (E. J. Robertson, ed., IRL Press Ltd. 1987); Guide to Techniques in Mouse Development (P. M. Wasserman et al. eds., Academic Press 1993); Embryonic Stem Cell Differentiation in vitro (M. V. Wiles, Meth. Enzymol. 225:900, 1993); Properties and uses of Embryonic Stem Cells: Prospects for Application to Human Biology and Gene Therapy (P. D. Rathjen et al., Reprod. Fertil. Dev. 10:31, 1998); and in Stem cell biology (L. M. Reid, Curr. Opinion Cell Biol. 2:121, 1990), each of which is incorporated by reference herein in its entirety.

### CITED REFERENCES

The following are a list of references cited in the detailed description above. Each of the following references is hereby incorporated by reference in its entirety and for all purposes.
1. Van Pham, P., et al., Isolation and proliferation of umbilical cord tissue derived mesenchymal stem cells for clinical applications. Cell Tissue Bank, 2015.
2. Fazzina, R., et al., A new standardized clinical-grade protocol for banking human umbilical cord tissue cells. Transfusion, 2015. 55(12): p. 2864-73.
3. Bieback, K., Platelet lysate as replacement for fetal bovine serum in mesenchymal stromal cell cultures. Transfus Med Hemother, 2013. 40(5): p. 326-35.
4. Stanko, P., et al., Comparison of human mesenchymal stem cells derived from dental pulp, bone marrow, adipose tissue, and umbilical cord tissue by gene expression.
5. Biomed Pap Med Fac Univ Palacky Olomouc Czech Repub, 2014. 158(3): p. 373-7. Schira, J., et al., Significant clinical, neuropathological and behavioural recovery from acute spinal cord trauma by transplantation of a well-defined somatic stem cell from human umbilical cord blood. Brain, 2012. 135(Pt 2): p. 431-46.
6. Hartmann, I., et al., Umbilical cord tissue-derived mesenchymal stem cells grow best under GMP-compliant culture conditions and maintain their phenotypic and functional properties. J Immunol Methods, 2010. 363(1): p. 80-9.
7. Friedman, R., et al., Umbilical cord mesenchymal stem cells: adjuvants for human cell transplantation. Biol Blood Marrow Transplant, 2007. 13(12): p. 1477-86.
8. Yu, B., et al., Enhanced mesenchymal stem cell survival induced by GATA-4 overexpression is partially mediated by regulation of the miR-15 family. Int J Biochem Cell Biol, 2013. 45(12): p. 2724-35.
9. Xu, W., et al., Basic fibroblast growth factor expression is implicated in mesenchymal stem cells response to light-induced retinal injury. Cell Mol Neurobiol, 2013. 33(8): p. 1171-9.
10. Fang, Z., et al., Differentiation of GFP-Bcl-2-engineered mesenchymal stem cells towards a nucleus pulposus-like phenotype under hypoxia in vitro. Biochem Biophys Res Commun, 2013. 432(3): p. 444-50.
11. Li, W., et al., Bcl-2 engineered MSCs inhibited apoptosis and improved heart function. Stem Cells, 2007. 25(8): p. 2118-27.
12. Tsubokawa, T., et al., Impact of anti-apoptotic and anti-oxidative effects of bone marrow mesenchymal stem cells with transient overexpression of heme oxygenase-1 on myocardial ischemia. Am J Physiol Heart Circ Physiol, 2010. 298(5): p. H1320-9.
13. Zhou, H., et al., Exendin-4 protects adipose-derived mesenchymal stem cells from apoptosis induced by hydrogen peroxide through the PI3K/Akt-Sfrp2 pathways. Free Radic Biol Med, 2014. 77: p. 363-75.
14. Le Blanc, K., et al., Treatment of severe acute graft-versus-host disease with third party haploidentical mesenchymal stem cells. Lancet, 2004. 363(9419): p. 1439-41.
15. Lazarus, H. M., et al., Cotransplantation of HLA-identical sibling culture-expanded mesenchymal stem cells and hematopoietic stem cells in hematologic malignancy patients. Biol Blood Marrow Transplant, 2005. 11(5): p. 389-98.
16. Bernardo, M. E., et al., Optimization of in vitro expansion of human multipotent mesenchymal stromal cells for cell-therapy approaches: further insights in the search for a fetal calf serum substitute. J Cell Physiol, 2007. 211(1): p. 121-30.
17. Reinisch, A., et al., Humanized system to propagate cord blood-derived multipotent mesenchymal stromal cells for clinical application. Regen Med, 2007. 2(4): p. 371-82.
18. Capelli, C., et al., Human platelet lysate allows expansion and clinical grade production of mesenchymal stromal cells from small samples of bone marrow aspirates or marrow filter washouts. Bone Marrow Transplant, 2007. 40(8): p. 785-91.
19. Lataillade, J. J., et al., New approach to radiation burn treatment by dosimetry-guided surgery combined with autologous mesenchymal stem cell therapy. Regen Med, 2007. 2(5): p. 785-94.
Seshareddy, K., D. Troyer, and M. L. Weiss, Method to isolate mesenchymal-like cells from Wharton's Jelly of umbilical cord. Methods Cell Biol, 2008. 86: p. 101-19.
21. Sensebe, L., Clinical grade production of mesenchymal stem cells. Biomed Mater Eng, 2008. 18(1 Suppl): p. S3-10.
22. Sotiropoulou, P. A., S. A. Perez, and M. Papamichail, Clinical grade expansion of human bone marrow mesenchymal stem cells. Methods Mol Biol, 2007. 407: p. 245-63.
23. Shetty, P., et al., Clinical grade mesenchymal stem cells transdifferentiated under xenofree conditions alleviates motor deficiencies in a rat model of Parkinson's disease. Cell Biol Int, 2009. 33(8): p. 830-8.
24. Zhang, X., et al., Cotransplantation of HLA-identical mesenchymal stem cells and hematopoietic stem cells in Chinese patients with hematologic diseases. Int J Lab Hematol, 2010. 32(2): p. 256-64.
Arrigoni, E., et al., Isolation, characterization and osteogenic differentiation of adipose-derived stem cells: from small to large animal models. Cell Tissue Res, 2009. 338(3): p. 401-11.
26. Grisendi, G., et al., GMP-manufactured density gradient media for optimized mesenchymal stromal/stem cell isolation and expansion. Cytotherapy, 2010. 12(4): p. 466-77.
27. Prasad, V. K., et al., Efficacy and safety of ex vivo cultured adult human mesenchymal stem cells (Prochymal) in pediatric patients with severe refractory acute graft-versus-host disease in a compassionate use study. Biol Blood Marrow Transplant, 2011. 17(4): p. 534-41.
28. Sensebe, L., P. Bourin, and K. Tarte, Good manufacturing practices production of mesenchymal stem/stromal cells. Hum Gene Ther, 2011. 22(1): p. 19-26.
29. Capelli, C., et al., Minimally manipulated whole human umbilical cord is a rich source of clinical-grade human mesenchymal stromal cells expanded in human platelet lysate. Cytotherapy, 2011. 13(7): p. 786-801.
30. Ilic, N., et al., Manufacture of clinical grade human placenta-derived multipotent mesenchymal stromal cells. Methods Mol Biol, 2011. 698: p. 89-106.
31. Santos, F., et al., Toward a clinical-grade expansion of mesenchymal stem cells from human sources: a microcarrier-based culture system under xeno-free conditions. Tissue Eng Part C Methods, 2011. 17(12): p. 1201-10.
32. Timmins, N. E., et al., Closed system isolation and scalable expansion of human placental mesenchymal stem cells. Biotechnol Bioeng, 2012. 109(7): p. 1817-26.
33. Warnke, P. H., et al., A clinically feasible protocol for using human platelet lysate and mesenchymal stem cells in regenerative therapies. J Craniomaxillofac Surg, 2013. 41(2): p. 153-61.
34. Fekete, N., et al., GMP-compliant isolation and large-scale expansion of bone marrow-derived MSC. PLoS One, 2012. 7(8): p. e43255.
35. Hanley, P. J., et al., Manufacturing mesenchymal stromal cells for phase I clinical trials. Cytotherapy, 2013. 15(4): p. 416-22.
36. Trojahn Kolle, S. F., et al., Pooled human platelet lysate versus fetal bovine serum-investigating the proliferation rate, chromosome stability and angiogenic potential of human adipose tissue-derived stem cells intended for clinical use. Cytotherapy, 2013. p. 1086-97.
37. Veronesi, E., et al., Transportation conditions for prompt use of ex vivo expanded and freshly harvested clinical-grade bone marrow mesenchymal stromal/stem cells for bone regeneration. Tissue Eng Part C Methods, 2014. 20(3): p. 239-51.
38. Dolley-Sonneville, P. J., L. E. Romeo, and Z. K. Melkoumian, Synthetic surface for expansion of human mesenchymal stem cells in xeno-free, chemically defined culture conditions. PLoS One, 2013. 8(8): p. e70263.
39. Siciliano, C., et al., Optimization of the isolation and expansion method of human mediastinal-adipose tissue derived mesenchymal stem cells with virally inactivated GMP-grade platelet lysate. Cytotechnology, 2015. 67(1): p. 165-74.
40. Martins, J. P., et al., Towards an advanced therapy medicinal product based on mesenchymal stromal cells isolated from the umbilical cord tissue: quality and safety data. Stem Cell Res Ther, 2014. 5(1): p. 9.
41. Iudicone, P., et al., Pathogen-free, plasma poor platelet lysate and expansion of human mesenchymal stem cells. J Transl Med, 2014. 12: p. 28.
42. Skrahin, A., et al., Autologous mesenchymal stromal cell infusion as adjunct treatment in patients with multidrug and extensively drug-resistant tuberculosis: an open-label phase I safety trial. Lancet Respir Med, 2014. 2(2): p. 108-22.
43. Ikebe, C. and K. Suzuki, Mesenchymal stem cells for regenerative therapy: optimization of cell preparation protocols. Biomed Res Int, 2014. 2014: p. 951512.
44. Chatzistamatiou, T. K., et al., Optimizing isolation culture and freezing methods to preserve Wharton's jelly's mesenchymal stem cell (MSC) properties: an MSC banking protocol validation for the Hellenic Cord Blood Bank. Transfusion, 2014. 54(12): p. 3108-20.
Swamynathan, P., et al., Are serum free and xeno-free culture conditions ideal for large scale clinical grade expansion of Wharton's jelly derived mesenchymal stem cells? A comparative study. Stem Cell Res Ther, 2014. 5(4): p. 88.
46. Vaes, B., et al., Culturing protocols for human multipotent adult stem cells. Methods Mol Biol, 2015. 1235: p. 49-58.
47. Devito, L., et al., Wharton's jelly mesenchymal stromal/stem cells derived under chemically defined animal product free low oxygen conditions are rich in MSCA-1(+) subpopulation. Regen Med, 2014. 9(6): p. 723-32.
48. Nemunaitis, J., et al., Human marrow stromal cells: response to interleukin-6 (IL-6) and control of IL-6 expression. Blood, 1989. 74(6): p. 1929-35.
49. Sadovnikova, E. Y., et al., Induction of hematopoietic microenvironment by the extracellular matrix from long-term bone marrow cultures. Ann Hematol, 1991. 62(5): p. 160-4.
50. Lazarus, H. M., et al., Ex vivo expansion and subsequent infusion of human bone marrow-derived stromal progenitor cells (mesenchymal progenitor cells): implications for therapeutic use. Bone Marrow Transplant, 1995. 16(4): p. 557-64.
51. Yoo, J. U., et al., The chondrogenic potential of human bone-marrow-derived mesenchymal progenitor cells. J Bone Joint Surg Am, 1998. 80(12): p. 1745-57.
52. Fleming, J. E., Jr., et al., Monoclonal antibody against adult marrow-derived mesenchymal stem cells recognizes developing vasculature in embryonic human skin. Dev Dyn, 1998. 212(1): p. 119-32.
53. Ghilzon, R., C. A. McCulloch, and R. Zohar, Stromal mesenchymal progenitor cells. Leuk Lymphoma, 1999. 32(3-4): p. 211-21.
54. De Cesaris, V., et al., Isolation, proliferation and characterization of endometrial canine stem cells. Reprod Domest Anim, 2016.
55. Van Pham, P., et al., Isolation and proliferation of umbilical cord tissue derived mesenchymal stem cells for clinical applications. Cell Tissue Bank, 2016. 17(2): p. 289-302.
56. Zhao, G., et al., Large-scale expansion of Wharton's jelly-derived mesenchymal stem cells on gelatin microbeads, with retention of self-renewal and multipotency characteristics and the capacity for enhancing skin wound healing. Stem Cell Res Ther, 2015. 6: p. 38.
57. Huang, P., et al., Differentiation of human umbilical cord Wharton's jelly-derived mesenchymal stem cells into germ-like cells in vitro. J Cell Biochem, 2010. 109(4): p. 747-54.
58. Wang, S. J., et al., Chondrogenic Potential of Peripheral Blood Derived Mesenchymal Stem Cells Seeded on Demineralized Cancellous Bone Scaffolds. Sci Rep, 2016. 6: p. 36400.
59. Fazeli, Z., M. D. Omrani, and S. M. Ghaderian, CD29/CD184 expression analysis provides a signature for identification of neuronal like cells differentiated from PBMSCs. Neurosci Lett, 2016. 630: p. 189-93.
60. Wu, G., et al., Osteogenesis of peripheral blood mesenchymal stem cells in self assembling peptide nanofiber for healing critical size calvarial bony defect. Sci Rep, 2015. 5: p. 16681.
61. Shaer, A., et al., Isolation and characterization of Human Mesenchymal Stromal Cells Derived from Placental Decidua Basalis; Umbilical cord Wharton's Jelly and Amniotic Membrane. Pak J Med Sci, 2014. 30(5): p. 1022-6.
62. Fu, W. L., C. Y. Zhou, and J. K. Yu, A new source of mesenchymal stem cells for articular cartilage repair: MSCs derived from mobilized peripheral blood share similar biological characteristics in vitro and chondrogenesis in vivo as MSCs from bone marrow in a rabbit model. Am J Sports Med, 2014. 42(3): p. 592-601.
63. El-Badawy, A., et al., Adipose Stem Cells Display Higher Regenerative Capacities and More Adaptable Electro-Kinetic Properties Compared to Bone Marrow-Derived Mesenchymal Stromal Cells. Sci Rep, 2016. 6: p. 37801.
64. Plock, J. A., et al., The Influence of Timing and Frequency of Adipose-Derived Mesenchymal Stem Cell Therapy on Immunomodulation Outcomes After Vascularized Composite Allotransplantation. Transplantation, 2017. 101(1): p. el-ell.
65. Celermajer, D. S., et al., Non-invasive detection of endothelial dysfunction in children and adults at risk of atherosclerosis. Lancet, 1992. 340(8828): p. 1111-5.
66. Palmer, R. M., A. G. Ferrige, and S. Moncada, Nitric oxide release accounts for the biological activity of endothelium-derived relaxing factor. Nature, 1987. 327(6122): p. 524-6.
67. Ahlers, B. A., et al., An age-related decline in endothelial function is not associated with alterations in L-arginine transport in humans. J Hypertens, 2004. 22(2): p. 321-7.
68. Taddei, S., et al., Aging and endothelial function in normotensive subjects and patients with essential hypertension. Circulation, 1995. 91(7): p. 1981-7.
69. Andrawis, N., D. S. Jones, and D. R. Abernethy, Aging is associated with endothelial dysfunction in the human forearm vasculature. J Am Geriatr Soc, 2000. 48(2): p. 193-8.
70. Ghiadoni, L., et al., Endothelial dysfunction and oxidative stress in chronic renal failure. J Nephrol, 2004. 17(4): p. 512-9.
71. Bilsborough, W., et al., Anti-tumour necrosis factor-alpha therapy over conventional therapy improves endothelial function in adults with rheumatoid arthritis. Rheumatol Int, 2006. 26(12): p. 1125-31.
72. Roifman, I., et al., Evidence of endothelial dysfunction in patients with inflammatory bowel disease. Clin Gastroenterol Hepatol, 2009. 7(2): p. 175-82.
73. Hurks, R., et al., Early endothelial dysfunction in young type 1 diabetics. Eur J Vasc Endovasc Surg, 2009. 37(5): p. 611-5.
74. Crisby, M., et al., Circulating levels of autoantibodies to oxidized low-density lipoprotein and C-reactive protein levels correlate with endothelial function in resistance arteries in men with coronary heart disease. Heart Vessels, 2009. 24(2): p.
75. Dede, D. S., et al., Assessment of endothelial function in Alzheimer's disease: is Alzheimer's disease a vascular disease? J Am Geriatr Soc, 2007. 55(10): p. 1613-7.
76. Chong, A. Y., et al., Endothelial dysfunction and damage in congestive heart failure: relation of flow-mediated dilation to circulating endothelial cells, plasma indexes of endothelial damage, and brain natriuretic peptide. Circulation, 2004. 110(13): p. 1794-8.
77. Poredos, P., Endothelial dysfunction in the pathogenesis of atherosclerosis. Int Angiol, 2002. 21(2): p. 109-16.
78. Listi, F., et al., PECAM-1/CD31 in infarction and longevity. Ann N Y Acad Sci, 2007. 1100: p. 132-9.
79. Ballard, V. L. and J. M. Edelberg, Targets for regulating angiogenesis in the ageing endothelium. Expert Opin Ther Targets, 2007. 11(11): p. 1385-99.
80. Lu, C., et al., Effect of age on vascularization during fracture repair. J Orthop Res, 2008. 26(10): p. 1384-9.
81. Rivard, A., et al., Age-dependent defect in vascular endothelial growth factor expression is associated with reduced hypoxia-inducible factor 1 activity. J Biol Chem, 2000. 275(38): p. 29643-7.
82. Herrmann, J. and A. Lerman, The Endothelium—the Cardiovascular Health Barometer. Herz, 2008. 33(5): p. 343-353.
83. Hamel, E., Perivascular nerves and the regulation of cerebrovascular tone. J Appl Physiol, 2006. 100(3): p. 1059-64.
84. Saenz de Tejada, I., et al., Pathophysiology of erectile dysfunction. J Sex Med, 2005. 2(1): p. 26-39.
85. Provis, J. M., et al., Anatomy and development of the macula: specialisation and the vulnerability to macular degeneration. Clin Exp Optom, 2005. 88(5): p. 269-81.
86. Izikki, M., et al., Role for dysregulated endothelium-derived FGF2 signaling in progression of pulmonary hypertension. Rev Mal Respir, 2008. 25(9): p. 1192.
87. Pautler, E. L., The possible role and treatment of deficient microcirculation regulation in age-associated memory impairment. Med Hypotheses, 1994. 42(6): p. 363-6.
88. McCarron, R. M., et al., Endothelial-mediated regulation of cerebral microcirculation. J Physiol Pharmacol, 2006. 57 Suppl 11: p. 133-44.
89. Nowak, J. Z., Age-related macular degeneration (AMD): pathogenesis and therapy. Pharmacol Rep, 2006. 58(3): p. 353-63.
90. Chai, S. J., E. Barrett-Connor, and A. Gamst, Small-vessel lower extremity arterial disease and erectile dysfunction: The Rancho Bernardo study. Atherosclerosis, 2009. 203(2): p. 620-5.
91. Tuder, R. M. and J. H. Yun, Vascular endothelial growth factor of the lung: friend or foe. Curr Opin Pharmacol, 2008. 8(3): p. 255-60.
92. Stump, M. M., et al., Endothelium Grown from Circulating Blood on Isolated Intravascular Dacron Hub. Am J Pathol, 1963. 43: p. 361-7.
93. Asahara, T., et al., Isolation of putative progenitor endothelial cells for angiogenesis. Science, 1997. 275(5302): p. 964-7.
94. Takahashi, T., et al., Ischemia-and cytokine-induced mobilization of bone marrow-derived endothelial progenitor cells for neovascularization. Nat Med, 1999. 5(4): p. 434-8.
Asahara, T., et al., Bone marrow origin of endothelial progenitor cells responsible for postnatal vasculogenesis in physiological and pathological neovascularization. Circ Res, 1999. 85(3): p. 221-8.
96. Peichev, M., et al., Expression of VEGFR-2 and AC133 by circulating human CD34(+) cells identifies a population of functional endothelial precursors. Blood, 2000. 95(3): p. 952-8.
97. Korbling, M., et al., Recombinant human granulocyte-colony-stimulating factor-mobilized and apheresis-collected endothelial progenitor cells: a novel blood cell component for therapeutic vasculogenesis. Transfusion, 2006. 46(10): p. 1795-802.
98. Timmermans, F., et al., Endothelial outgrowth cells are not derived from CD133+ cells or CD45+ hematopoietic precursors. Arterioscler Thromb Vasc Biol, 2007. 27(7): p. 1572-9.
99. Rehman, J., et al., Peripheral blood “endothelial progenitor cells” are derived from monocyte/macrophages and secrete angiogenic growth factors. Circulation, 2003. 107(8): p. 1164-9.
100. Rohde, E., et al., Blood monocytes mimic endothelial progenitor cells. Stem Cells, 2006. 24(2): p. 357-67.
101. Foteinos, G., et al., Rapid endothelial turnover in atherosclerosis prone areas coincides with stem cell repair in apolipoprotein E-deficient mice. Circulation, 2008. 117(14): p. 1856-63.
102. Werner, N., et al., Intravenous transfusion of endothelial progenitor cells reduces neointima formation after vascular injury. Circ Res, 2003. 93(2): p. e17-24.
103. Wassmann, S., et al., Improvement of endothelial function by systemic transfusion of vascular progenitor cells. Circ Res, 2006. 99(8): p. e74-83.
104. Edelberg, J. M., et al., Young adult bone marrow-derived endothelial precursor cells restore aging-impaired cardiac angiogenic function. Circ Res, 2002. 90(10): p. E89-93.
105. Hristov, M., et al., Adult progenitor cells in vascular remodeling during atherosclerosis. Biol Chem, 2008. 389(7): p. 837-44.
106. Zhao, Q., et al., Stem/progenitor cells in liver injury repair and regeneration. Biol Cell, 2009. 101(10): p. 557-71.
107. Sun, Y., Myocardial repair/remodelling following infarction: roles of local factors. Cardiovasc Res, 2009. 81(3): p. 482-90.
108. Ogami, M., et al., Telomere shortening in human coronary artery diseases. Arterioscler Thromb Vasc Biol, 2004. 24(3): p. 546-50.
109. Goldschmidt-Clermont, P. J., Loss of bone marrow-derived vascular progenitor cells leads to inflammation and atherosclerosis. Am Heart J, 2003. 146(4 Suppl): p. S5-12.
110. Spyridopoulos, I., et al., Telomere gap between granulocytes and lymphocytes is a determinant for hematopoetic progenitor cell impairment in patients with previous myocardial infarction. Arterioscler Thromb Vasc Biol, 2008. 28(5): p. 968-74.
111. Griese, D. P., et al., Isolation and transplantation of autologous circulating endothelial cells into denuded vessels and prosthetic grafts: implications for cell-based vascular therapy. Circulation, 2003. 108(21): p. 2710-5.
112. Liu, F., et al., Transplanted endothelial progenitor cells ameliorate carbon tetrachloride-induced liver cirrhosis in rats. Liver Transpl, 2009. 15(9): p. 1092-100.
113. Nakamura, T., et al., Significance and therapeutic potential of endothelial progenitor cell transplantation in a cirrhotic liver rat model. Gastroenterology, 2007. 133(1): p. 91-107 el.
114. Xin, Z., et al., Different biological properties of circulating and bone marrow endothelial progenitor cells in acute myocardial infarction rats. Thorac Cardiovasc Surg, 2008. 56(8): p. 441-8.
115. Vila, V., et al., Inflammation, endothelial dysfunction and angiogenesis markers in chronic heart failure patients. Int J Cardiol, 2008. 130(2): p. 276-7.
116. von Haehling, S., et al., Inflammatory biomarkers in heart failure revisited: much more than innocent bystanders. Heart Fail Clin, 2009. 5(4): p. 549-60.
117. Stenvinkel, P., Inflammation in end-stage renal disease—a fire that burns within. Contrib Nephrol, 2005. 149: p. 185-99.
118. Porazko, T., et al., IL-18 is involved in vascular injury in end-stage renal disease patients. Nephrol Dial Transplant, 2009. 24(2): p. 589-96.
119. Nakae, H., et al., Involvement of IL-18 and soluble fas in patients with postoperative hepatic failure. Eur Surg Res, 2003. 35(2): p. 61-6.
120. Yumoto, E., et al., Serum gamma-interferon-inducing factor (IL-18) and IL-10 levels in patients with acute hepatitis and fulminant hepatic failure. J Gastroenterol Hepatol, 2002. 17(3): p. 285-94.
121. Petrovic-Rackov, L. and N. Pejnovic, Clinical significance of IL-18, IL-15, IL-12 and TNF-alpha measurement in rheumatoid arthritis. Clin Rheumatol, 2006. 25(4): p. 448-52.
122. Leach, S. T., et al., Local and systemic interleukin-18 and interleukin-18-binding protein in children with inflammatory bowel disease. Inflamm Bowel Dis, 2008. 14(1): p. 68-74.
123. Miles, E. A., et al., Age-related increases in circulating inflammatory markers in men are independent of BMI, blood pressure and blood lipid concentrations. Atherosclerosis, 2008. 196(1): p. 298-305.
124. Krabbe, K. S., M. Pedersen, and H. Bruunsgaard, Inflammatory mediators in the elderly. Exp Gerontol, 2004. 39(5): p. 687-99.
125. Svoboda, P., et al., Neopterin, a marker of immune response, and 8-hydroxy-2′-deoxyguanosine, a marker of oxidative stress, correlate at high age as determined by automated simultaneous high-performance liquid chromatography analysis of human urine. Anal Biochem, 2008. 383(2): p. 236-42.
126. Blasko, I., et al., Cognitive deterioration in Alzheimer's disease is accompanied by increase of plasma neopterin. J Psychiatr Res, 2007. 41(8): p. 694-701.
127. Capri, M., et al., The genetics of human longevity. Ann N Y Acad Sci, 2006. 1067: p. 252-63.
128. Ventura, E., et al., Homocysteine and inflammation as main determinants of oxidative stress in the elderly. Free Radic Biol Med, 2008.
129. van Leuven, S. I., et al., ApoAI-phosphatidylcholine infusion neutralizes the atherothrombotic effects of C-reactive protein in humans. J Thromb Haemost, 2008.
130. Nagaoka, T., et al., C-reactive protein inhibits endothelium-dependent nitric oxide-mediated dilation of retinal arterioles via enhanced superoxide production. Invest Ophthalmol Vis Sci, 2008. 49(5): p. 2053-60.
131. Butovsky, O., et al., Induction and blockage of oligodendrogenesis by differently activated microglia in an animal model of multiple sclerosis. J Clin Invest, 2006. 116(4): p. 905-15.
132. Pickering, M. and J. J. O'Connor, Pro-inflammatory cytokines and their effects in the dentate gyrus. Prog Brain Res, 2007. 163: p. 339-54.
133. Pluchino, S., et al., Persistent inflammation alters the function of the endogenous brain stem cell compartment. Brain, 2008. 131(Pt 10): p. 2564-78.
134. Fiorito, C., et al., Antioxidants increase number of progenitor endothelial cells through multiple gene expression pathways. Free Radic Res, 2008. 42(8): p. 754-62.
135. Ablin, J. N., et al., Effect of anti-TNFalpha treatment on circulating endothelial progenitor cells (EPCs) in rheumatoid arthritis. Life Sci, 2006. 79(25): p. 2364-9.
136. Bosello, S., et al., TNF-alpha blockade induces a reversible but transient effect on endothelial dysfunction in patients with long-standing severe rheumatoid arthritis. Clin Rheumatol, 2008. 27(7): p. 833-9.
137. Garcia-Olmo, D., et al., A phase I clinical trial of the treatment of Crohn's fistula by adipose mesenchymal stem cell transplantation. Dis Colon Rectum, 2005. 48(7): p. 1416-23.
138. Stillaert, F. B., et al., Human clinical experience with adipose precursor cells seeded on hyaluronic acid-based spongy scaffolds. Biomaterials, 2008. 29(29): p. 3953-9.
139. Garcia-Olmo, D., M. Garcia-Arranz, and D. Herreros, Expanded adipose-derived stem cells for the treatment of complex perianal fistula including Crohn's disease. Expert Opin Biol Ther, 2008. 8(9): p. 1417-23.
140. Fang, B., et al., Treatment of severe therapy-resistant acute graft-versus-host disease with human adipose tissue-derived mesenchymal stem cells. Bone Marrow Transplant, 2006. 38(5): p. 389-90.
141. Fang, B., et al., Using human adipose tissue-derived mesenchymal stem cells as salvage therapy for hepatic graft-versus-host disease resembling acute hepatitis. Transplant Proc, 2007. 39(5): p. 1710-3.
142. Fang, B., et al., Favorable response to human adipose tissue-derived mesenchymal stem cells in steroid-refractory acute graft-versus-host disease. Transplant Proc, 2007. 39(10): p. 3358-62.
143. Hayashi, O., et al., Comparison of osteogenic ability of rat mesenchymal stem cells from bone marrow, periosteum, and adipose tissue. Calcif Tissue Int, 2008. 82(3): p. 238-47.
144. Noel, D., et al., Cell specific differences between human adipose-derived and mesenchymal-stromal cells despite similar differentiation potentials. Exp Cell Res, 2008. 314(7): p. 1575-84.
145. Kim, Y., et al., Direct comparison of human mesenchymal stem cells derived from adipose tissues and bone marrow in mediating neovascularization in response to vascular ischemia. Cell Physiol Biochem, 2007. 20(6): p. 867-76.
146. Keyser, K. A., K. E. Beagles, and H. P. Kiem, Comparison of mesenchymal stem cells from different tissues to suppress T-cell activation. Cell Transplant, 2007. 16(5): p. 555-62.
147. Lin, K., et al., Characterization of adipose tissue-derived cells isolated with the Celution system. Cytotherapy, 2008. 10(4): p. 417-26.
148. http://www.tissuegenesis.com/TGI %201000%20Product %20Brochure.pdf.
149. Hollenberg, C. H. and A. Vost, Regulation of DNA synthesis in fat cells and stromal elements from rat adipose tissue. J Clin Invest, 1969. 47(11): p. 2485-98.
150. Gaben-Cogneville, A. M., et al., Differentiation under the control of insulin of rat preadipocytes in primary culture. Isolation of homogeneous cellular fractions by gradient centrifugation. Biochim Biophys Acta, 1983. 762(3): p. 437-44.
151. Glick, J. M. and S. J. Adelman, Established cell lines from rat adipose tissue that secrete lipoprotein lipase. In Vitro, 1983. 19(5): p. 421-8.
152. Zuk, P. A., et al., Multilineage cells from human adipose tissue: implications for cell-based therapies. Tissue Eng, 2001. 7(2): p. 211-28.
153. Zuk, P. A., et al., Human adipose tissue is a source of multipotent stem cells. Mol Biol Cell, 2002. 13(12): p. 4279-95.
154. Prockop, D. J., Marrow stromal cells as stem cells for nonhematopoietic tissues. Science, 1997. 276(5309): p. 71-4.
155. Meliga, E., et al., Adipose-derived cells. Cell Transplant, 2007. 16(9): p. 963-70.
156. http://www.cytoritx.com/intl/products/cv_clinical_trials.html.
157. Fang, B., et al., Human adipose tissue-derived mesenchymal stromal cells as salvage therapy for treatment of severe refractory acute graft-vs.-host disease in two children. Pediatr Transplant, 2007. 11(7): p. 814-7.
158. Ichim, T. E., et al., Placental mesenchymal and cord blood stem cell therapy for dilated cardiomyopathy. Reprod Biomed Online, 2008. 16(6): p. 898-905.
159. Kim, S. W., et al., Successful stem cell therapy using umbilical cord blood-derived multipotent stem cells for Buerger's disease and ischemic limb disease animal model. Stem Cells, 2006. 24(6): p. 1620-6.
160. Almeida-Porada, G., et al., Cotransplantation of human stromal cell progenitors into preimmune fetal sheep results in early appearance of human donor cells in circulation and boosts cell levels in bone marrow at later time points after transplantation. Blood, 2000. 95(11): p. 3620-7.
161. Noort, W. A., et al., Mesenchymal stem cells promote engraftment of human umbilical cord blood-derived CD34(+) cells in NOD/SCID mice. Experimental hematology, 2002. 30(8): p. 870-8.
162. Lazarus, H. M., et al., Ex vivo expansion and subsequent infusion of human bone marrow-derived stromal progenitor cells (mesenchymal progenitor cells): implications for therapeutic use. Bone marrow transplantation, 1995. 16(4): p. 557-64.
163. Koc, O. N., et al., Rapid hematopoietic recovery after coinfusion of autologous-blood stem cells and culture-expanded marrow mesenchymal stem cells in advanced breast cancer patients receiving high-dose chemotherapy. Journal of clinical oncology: official journal of the American Society of Clinical Oncology, 2000. 18(2): p. 307-16.
164. Lazarus, H. M., et al., Cotransplantation of HLA-identical sibling culture-expanded mesenchymal stem cells and hematopoietic stem cells in hematologic malignancy patients. Biology of blood and marrow transplantation: journal of the American Society for Blood and Marrow Transplantation, 2005. 11(5): p. 389-98.
165. Oka, T., et al., Evidence for specific immune response against P210 BCR-ABL in long-term remission CML patients treated with interferon. Leukemia: official journal of the Leukemia Society of America, Leukemia Research Fund, U. K, 1998. 12(2): p. 155-63.
166. Pulsipher, M. A., et al., Allogeneic transplantation for pediatric acute lymphoblastic leukemia: the emerging role of peritransplantation minimal residual disease/chimerism monitoring and novel chemotherapeutic, molecular, and immune approaches aimed at preventing relapse. Biology of blood and marrow transplantation: journal of the American Society for Blood and Marrow Transplantation, 2009. 15(1 Suppl): p. 62-71.
167. Griffin, M. D., T. Ritter, and B. P. Mahon, Immunological aspects of allogeneic mesenchymal stem cell therapies. Human gene therapy, 2010. 21(12): p. 1641-55.
168. Xue, Q., et al., The negative co-signaling molecule b7-h4 is expressed by human bone marrow-derived mesenchymal stem cells and mediates its T-cell modulatory activity. Stem cells and development, 2010. 19(1): p. 27-38.
169. Ruhnke, M., et al., Differentiation of in vitro-modified human peripheral blood monocytes into hepatocyte-like and pancreatic islet-like cells. Gastroenterology, 2005. 128(7): p. 1774-86.
170. Ruhnke, M., et al., Human monocyte-derived neohepatocytes: a promising alternative to primary human hepatocytes for autologous cell therapy. Transplantation, 2005. 79(9): p. 1097-103.
171. Suganami, T., J. Nishida, and Y. Ogawa, A paracrine loop between adipocytes and macrophages aggravates inflammatory changes: role of free fatty acids and tumor necrosis factor alpha. Arterioscler Thromb Vasc Biol, 2005. 25(10): p. 2062-8.
172. Bastard, J. P., et al., Recent advances in the relationship between obesity, inflammation, and insulin resistance. Eur Cytokine Netw, 2006. 17(1): p. 4-12.
173. Zeyda, M. and T. M. Stulnig, Adipose tissue macrophages. Immunol Lett, 2007. 112(2): p. 61-7.
174. Odegaard, J. I., et al., Macrophage-specific PPARgamma controls alternative activation and improves insulin resistance. Nature, 2007. 447(7148): p. 1116-20.
175. Zeyda, M., et al., Human adipose tissue macrophages are of an anti-inflammatory phenotype but capable of excessive pro-inflammatory mediator production. Int J Obes (Lond), 2007. 31(9): p. 1420-8.
176. Mantovani, A., et al., Macrophage polarization: tumor-associated macrophages as a paradigm for polarized M2 mononuclear phagocytes. Trends Immunol, 2002. 23(11): p. 549-55.
177. Mehta, A., et al., Infection-induced modulation of m1 and m2 phenotypes in circulating monocytes: role in immune monitoring and early prognosis of sepsis. Shock, 2004. 22(5): p. 423-30.
178. Song, G. Y., et al., Evolution of an immune suppressive macrophage phenotype as a product of P38 MAPK activation in polymicrobial sepsis. Shock, 2001. 15(1): p. 42-8.
179. Gustafsson, C., et al., Gene expression profiling of human decidual macrophages: evidence for immunosuppressive phenotype. PLoS ONE, 2008. 3(4): p. e2078.
180. Dong, Z., et al., Stromal vascular fraction (SVF) cells enhance long-term survival of autologous fat grafting through the facilitation of M2 macrophages. Cell Biol Int, 2013.
181. Astori, G., et al., “In vitro” and multicolor phenotypic characterization of cell subpopulations identified in fresh human adipose tissue stromal vascular fraction and in the derived mesenchymal stem cells. J Transl Med, 2007. 5: p. 55.
182. Wang, Y., et al., Ex vivo programmed macrophages ameliorate experimental chronic inflammatory renal disease. Kidney Int, 2007. 72(3): p. 290-9.
183. Ponomarev, E. D., et al., CNS-derived interleukin-4 is essential for the regulation of autoimmune inflammation and induces a state of alternative activation in microglial cells. J Neurosci, 2007. 27(40): p. 10714-21.
184. Zhang, X., et al., Generation of therapeutic dendritic cells and regulatory T cells for preventing allogeneic cardiac graft rejection. Clin Immunol, 2008. 127(3): p. 313-21.
185. Ichim, T. E., R. Zhong, and W. P. Min, Prevention of allograft rejection by in vitro generated tolerogenic dendritic cells. Transpl Immunol, 2003. 11(3-4): p. 295-306.
186. Tiemessen, M. M., et al., CD4+CD25+Foxp3+ regulatory T cells induce alternative activation of human monocytes/macrophages. Proc Natl Acad Sci USA, 2007. 104(49): p. 19446-51.
187. Ryan, J. M., et al., Interferon-gamma does not break, but promotes the immunosuppressive capacity of adult human mesenchymal stem cells. Clin Exp Immunol, 2007. 149(2): p. 353-63.
188. Ye, Z., et al., Immunosuppressive effects of rat mesenchymal stem cells: involvement of CD4+CD25+ regulatory T cells. Hepatobiliary Pancreat Dis Int, 2008. 7(6): p. 608-14.
189. Askenasy, N., A. Kaminitz, and S. Yarkoni, Mechanisms of T regulatory cell function. Autoimmun Rev, 2008. 7(5): p. 370-5.
190. Gonzalez-Rey, E., et al., Human adipose-derived mesenchymal stem cells reduce inflammatory and T-cell responses and induce regulatory T cells in vitro in rheumatoid arthritis. Ann Rheum Dis, 2009.
191. Casiraghi, F., et al., Pretransplant infusion of mesenchymal stem cells prolongs the survival of a semiallogeneic heart transplant through the generation of regulatory T cells. J Immunol, 2008. 181(6): p. 3933-46.
192. Di Ianni, M., et al., Mesenchymal cells recruit and regulate T regulatory cells. Exp Hematol, 2008. 36(3): p. 309-18.
193. Basak, G. W., et al., Human embryonic stem cells hemangioblast express HLA-antigens. J Transl Med, 2009. 7: p. 27.

### Example Embodiments

The following are example embodiments (e.g., example systems, methods, and computer-readable media) for increasing the quality of organs for transplantation. The examples are not intended to limit the implementations described herein but are intended to illustrate various embodiments.
1. A method of increasing the quality of organs for transplantation comprising the steps of: a) obtaining a brain-dead patient; b) maintaining viability of said brain dead patient by one or more life supporting technologies; c) administration of regenerative cells and; d) harvesting said organs.
2. The method of example embodiment 1, wherein said brain-dead patient is defined as a patient possessing clinical features of death
3A. The method of example embodiment 2, wherein said clinical features of death are selected from a group comprising of: a) unresponsive coma; b) absence of reflexes and c) and lack of any movements.
3B. The method of example embodiment 2, wherein said clinical features of death are present for after 1 hour of observation.
4. The method of example embodiment 1, wherein said brain death is defined as absence of breath after 3 min without mechanical ventilation.
5. The method of example embodiment 1, wherein said brain death is defined as an isoelectric EEG.
6. The method of example embodiments 2-5 wherein said patient was not exposed to hyperthermia.
7. The method of example embodiment 6, wherein said hyperthermia is below 32° C.
8. The method of example embodiments 2-5, wherein said patient was not under the influence of central nervous system depressants.
9. The method of example embodiment 2-5, wherein said patient undergoes repetition of clinical tests within 24 h of presentation.
10. The method of example embodiment 1, wherein said life supporting technologies include chemical agents and maintaining hormonal homeostasis.
11. The method of example embodiment 1, wherein said regenerative cell is a stem cell, including possibility a pluripotent stem cell or a mesenchymal stem cell.
12. The method of example embodiment 11, wherein said pluripotent stem cells are selected from a group of cells comprising of: a) inducible pluripotent stem cells; b) somatic cell nuclear transfer derived stem cells; c) embryonic stem cells; and d) parthenogenic derived stem cells.
13. The method of example embodiment 11, wherein said pluripotent stem cells are exposed to inflammatory stress before being provided to the brain dead patient.
14. The method of example embodiment 13, wherein said inflammatory stress is exposure to a toll-like receptor.
15. The method of example embodiment 14, wherein said inducible pluripotent stem cell possesses markers selected from a group comprising of: CD10, CD13, CD44, CD73, CD90, PDGFr-alpha, PD-L2, and HLA-A,B,C and possesses ability to undergo at least 40 doublings in culture, while maintaining a normal karyotype upon passaging.
16. The method of example embodiment 15, wherein said inducible pluripotent stem cells express OCT4.
17. The method of example embodiment 12, wherein said parthenogenic stem cells wherein said parthenogenically derived stem cells are generated by addition of a calcium flux inducing agent to activate an oocyte followed by enrichment of cells expressing markers selected from a group comprising of SSEA-4, TRA 1-60 and TRA 1-81.
18. The method of example embodiment 12, wherein said somatic cell nuclear transfer derived stem cells possess a phenotype negative for SSEA-1 and positive for SSEA-3, SSEA-4, TRA-1-60, TRA-1-81, and alkaline phosphatase.
19. The method of example embodiment 11, wherein said mesenchymal stem cell are derived from tissue comprising a group selected from: a) Wharton's Jelly; b) bone marrow; c) peripheral blood; d) mobilized peripheral blood; e) endometrium; f) hair follicle; g) deciduous tooth; h) testicle; i) adipose tissue; j) skin; k) amniotic fluid; l) cord blood; m) omentum; n) muscle; o) amniotic membrane; o) periventricular fluid; and p) placental tissue.
20. The method of example embodiment 19, wherein said mesenchymal stem cells express a marker or plurality of markers selected from a group comprising of: STRO-1, CD90, CD73, CD105, CD54, CD106, HLA-I markers, vimentin, ASMA, collagen-1, fibronectin, LFA-3, ICAM-1, PECAM-1, P-selectin, L-selectin, CD49b/CD29, CD49c/CD29, CD49d/CD29, CD61, CD18, CD29, thrombomodulin, telomerase, CD10, CD13, STRO-2, VCAM-1, CD146, and THY-1.
21. The method of example embodiment 11, wherein said mesenchymal stem cells do not express substantial levels of HLA-DR, CD117, and CD45.
22. The method of example embodiment 11, wherein said mesenchymal stem cells express CD56.
23. The method of example embodiment 11, wherein said mesenchymal stem cell are activated by exposure to a toll like receptor agonist
24. The method of example embodiment 23, wherein said toll like receptor is TLR-1.
25. The method of example embodiment 23, wherein said toll like receptor is TLR-2.
26. The method of example embodiment 25, wherein said activator of TLR-2 is HKLM.
27. The method of example embodiment 23, wherein said toll like receptor is TLR-3.
28. The method of example embodiment 27, wherein said activator of TLR-3 is Poly:IC.
29. The method of example embodiment 23, wherein said toll like receptor is TLR-4.
30. The method of example embodiment 29, wherein said activator of TLR-4 is LPS.
31. The method of example embodiment 29, wherein said activator of TLR-4 is Buprenorphine.
32. The method of example embodiment 29, wherein said activator of TLR-4 is Carbamazepine.
33. The method of example embodiment 29 wherein said activator of TLR-4 is Fentanyl.
34. The method of example embodiment 29, wherein said activator of TLR-4 is Levorphanol.
35. The method of example embodiment 29, wherein said activator of TLR-4 is Methadone.
36. The method of example embodiment 29, wherein said activator of TLR-4 is Cocaine.
37. The method of example embodiment 29, wherein said activator of TLR-4 is Morphine.
38. The method of example embodiment 29, wherein said activator of TLR-4 is Oxcarbazepine.
39. The method of example embodiment 29, wherein said activator of TLR-4 is Oxycodone.
40. The method of example embodiment 29, wherein said activator of TLR-4 is Pethidine.
41. The method of example embodiment 29, wherein said activator of TLR-4 is Glucuronoxylomannan from Cryptococcus.
42. The method of example embodiment 29, wherein said activator of TLR-4 is Morphine-3-glucuronide.
43. The method of example embodiment 29, wherein said activator of TLR-4 is lipoteichoic acid.
44. The method of example embodiment 29, wherein said activator of TLR-4 is β-defensin 2.
45. The method of example embodiment 29, wherein said activator of TLR-4 is small molecular weight hyaluronic acid.
46. The method of example embodiment 29, wherein said activator of TLR-4 is fibronectin EDA.
47. The method of example embodiment 29, wherein said activator of TLR-4 is snapin.
48. The method of example embodiment 29, wherein said activator of TLR-4 is tenascin C.
49. The method of example embodiment 23, wherein said toll like receptor is TLR-5.
50. The method of example embodiment 49, wherein said activator of TLR-5 is flagellin.
51. The method of example embodiment 23, wherein said toll like receptor is TLR-6.
52. The method of example embodiment 51, wherein said activator of TLR-6 is FSL-1.
53. The method of example embodiment 23, wherein said toll like receptor is TLR-7.
54. The method of example embodiment 53, wherein said activator of TLR-7 is imiquimod.
55. The method of example embodiment 23, wherein said toll like receptor of TLR-8.
56. The method of example embodiment 55, wherein said activator of TLR8 is ssRNA40/LyoVec.
57. The method of example embodiment 23, wherein said toll like receptor of TLR-9.
58. The method of example embodiment 57, wherein said activator of TLR-9 is a CpG oligonucleotide.
59. The method of example embodiment 58, wherein said activator of TLR-9 is ODN2006.
60. The method of example embodiment 58, wherein said activator of TLR-9 is Agatolimod.
61. The method of example embodiment 1, wherein said regenerative cells are monocytes.
62. The method of example embodiment 61, wherein said regenerative cells are monocytes that have been treated with interleukin-10.
63. The method of example embodiment 61, wherein said regenerative cells are monocytes that have been exposed to hypoxia.
64. The method of example embodiment 61, wherein said regenerative cells are monocytes that have been exposed to HGF-1.
65. The method of example embodiment 61, wherein said regenerative cells are monocytes that have been exposed to FGF-1.
66. The method of example embodiment 61, wherein said regenerative cells are monocytes that have been exposed to FGF-2.
67. The method of example embodiment 61, wherein said regenerative cells are monocytes that have been exposed to carbon monoxide.
68. The method of example embodiment 61, wherein said regenerative cells are monocytes that have been exposed to xenon.
69. The method of example embodiment 61, wherein said regenerative cells are monocytes that have been exposed to argon.
70. The method of example embodiment 61, wherein said regenerative cells are monocytes that have been exposed to krypton.
71. The method of example embodiment 61, wherein said regenerative cells are monocytes that have been exposed to neon.
72. The method of example embodiment 61, wherein said regenerative cells are monocytes that have been exposed to PGE-2.
73. The method of example embodiment 61, wherein said regenerative cells are monocytes that have been exposed to TGF-beta.
74. The method of example embodiment 61, wherein said regenerative cells are monocytes that have been exposed to leukemia inhibitor factor.
75. The method of example embodiment 61, wherein said regenerative cells are monocytes that have been exposed to interleukin-4.
76. The method of example embodiment 61, wherein said regenerative cells are monocytes that have been exposed to interleukin-13.
77. The method of example embodiment 61, wherein said regenerative cells are monocytes that have been exposed to interleukin-17.
78. The method of example embodiment 61, wherein said regenerative cells are monocytes that have been exposed to human chorionic gonadotrophin.
79. The method of example embodiment 61, wherein said regenerative cells are monocytes that have been exposed to IV)G.
80. The method of example embodiment 61, wherein said regenerative cells are monocytes that have been exposed to M-CSF.
81. The method of example embodiment 61, wherein said regenerative cells are monocytes that have been exposed to GM-CSF.
82. The method of example embodiment 1, wherein said regenerative cells are T regulatory cells.
83. The method of example embodiment 82, wherein said T regulatory cells are generated from pluripotent stem cells.
84. The method of example embodiment 82, wherein said T regulatory cells are generated from hematopoietic stem cells.
85. The method of example embodiment 82, wherein said T regulatory cells are generated from umbilical cord blood stem cells.
86. The method of example embodiment 82, wherein said T regulatory cells are generated from peripheral blood stem cells.
87. The method of example embodiment 82, wherein said T regulatory cells are generated from naïve T cells.
88. The method of example embodiment 82, wherein said T regulatory cells express FoxP3.
89. The method of example embodiment 82, wherein said T regulatory cells express interleukin-10 receptor.
90. The method of example embodiment 82, wherein said T regulatory cells express CCR8.
91. The method of example embodiment 82, wherein said T regulatory cells express CD25.
92. The method of example embodiment 82, wherein said T regulatory cells express CTLA4.
93. The method of example embodiment 82, wherein said T regulatory cells express ICOS-1.
94. The method of example embodiment 82, wherein said T regulatory cells express membrane bound TGF-beta.
95. The method of example embodiment 82, wherein said T regulatory cells express Fas ligand.
96. The method of example embodiment 82, wherein said T regulatory cells express CD3.
97. The method of example embodiment 82, wherein said T regulatory cells express CD4.
98. The method of example embodiment 82, wherein said T regulatory cells express CD56.
99. The method of example embodiment 82, wherein said T regulatory cells express CD57.
100. The method of example embodiment 82, wherein said T regulatory cells express HLA-G.
101. The method of example embodiment 1, wherein said regenerative cell is an endothelial progenitor cell
102. The method of example embodiment 101, wherein said circulating endothelial progenitor cells express CD133.
103. The method of example embodiment 101, wherein said circulating endothelial progenitor cells express CD34.
104. The method of example embodiment 101, wherein said circulating endothelial progenitor cells express CD34 and CD133.
105. The method of example embodiment 101, wherein said circulating endothelial progenitor cells express CD133 but lack expression of CD38.
106. The method of example embodiment 101, wherein said circulating endothelial progenitor cells express CD133 and CD34 but lack expression of CD34.
107. The method of example embodiment 101, wherein said circulating endothelial progenitor cells express VEGF-receptor.
108. The method of example embodiment 101, wherein said circulating endothelial progenitor cells express EGF-receptor.
109. The method of example embodiment 101, wherein said circulating endothelial progenitor cells express VEGF-receptor and CD45.
110. The method of example embodiment 101, wherein said circulating endothelial progenitor cells express VEGF-receptor and CD34.
111. The method of example embodiment 101, wherein said circulating endothelial progenitor cells express VEGF-receptor and CD133.
112. The method of example embodiment 101, wherein said circulating endothelial progenitor cells express VEGF-receptor and c-met.
113. The method of example embodiment 101, wherein said circulating endothelial progenitor cells express VEGF-receptor and c-met.
114. The method of example embodiment 113, wherein said circulating endothelial progenitor cells multiply once every approximately 12-36 hours.
115. The method of example embodiment 113, wherein said circulating endothelial progenitor cells multiply once every approximately 17-30 hours.
116. The method of example embodiment 113, wherein said circulating endothelial progenitor cells multiply once every approximately 20-24 hours.
117. The method of example embodiment 113, wherein said circulating endothelial progenitor cells produce interleukin 1 beta at a concentration of 1-8 picograms per million cells when stimulated with 1 ng/ml of lipopolysaccharide.
118. The method of example embodiment 113, wherein said circulating endothelial progenitor cells produce interleukin 1 beta at a concentration of 5-7 picograms per million cells when stimulated with 1 ng/ml of lipopolysaccharide.
119. The method of example embodiment 113, wherein said circulating endothelial progenitor cells produce at a concentration of 7 picograms per million cells when stimulated with 1 ng/ml of lipopolysaccharide.
120. The method of example embodiment 113, wherein said circulating endothelial progenitor cells produce FGF-1 at a concentration of 9-88 picograms per million cells when stimulated with 1 ng/ml of lipopolysaccharide.
121. The method of example embodiment 113, wherein said circulating endothelial progenitor cells produce interleukin 1 beta at a concentration of 30-79 picograms per million cells when stimulated with 1 ng/ml of lipopolysaccharide.
122. The method of example embodiment 113, wherein said circulating endothelial progenitor cells produce interleukin 2 beta at a concentration of 10-1300 picograms per million cells when stimulated with 1 ng/ml of lipopolysaccharide.
123. The method of example embodiment 113, wherein said circulating endothelial progenitor cells produce interleukin 1 beta at a concentration of 40 picograms per million cells when stimulated with 1 ng/ml of lipopolysaccharide.
124. The method of example embodiment 113, wherein said circulating endothelial progenitor cells express IL-3 receptor.
125. The method of example embodiment 113, wherein said circulating endothelial progenitor cells express IL-5 receptor.
126. The method of example embodiment 113, wherein said circulating endothelial progenitor cells express IL-8 receptor.
127. The method of example embodiment 113, wherein said circulating endothelial progenitor cells express IL-10 receptor.
128. The method of example embodiment 113, wherein said circulating endothelial progenitor cells express IL-13 receptor.
129. The method of example embodiment 113, wherein said circulating endothelial progenitor cells express IL-17 receptor.
130. The method of example embodiment 113, wherein said circulating endothelial progenitor cells express IL-21 receptor.
131. The method of example embodiment 113, wherein said circulating endothelial progenitor cells express IL-33 receptor.
132. The method of example embodiment 113, wherein said circulating endothelial progenitor cells express interferon alpha receptor.
133. The method of example embodiment 113, wherein said circulating endothelial progenitor cells express interferon beta receptor.
134. The method of example embodiment 113, wherein said circulating endothelial progenitor cells express interferon gamma receptor.
135. The method of example embodiment 113, wherein said circulating endothelial progenitor cells express interferon omega receptor.
136. The method of example embodiment 113, wherein said circulating endothelial progenitor cells express CD11b.
137. The method of example embodiment 113, wherein said circulating endothelial progenitor cells express CD11c.
138. The method of example embodiment 113, wherein said circulating endothelial progenitor cells express CD14.
139. The method of example embodiment 113, wherein said circulating endothelial progenitor cells express CD40.
140. The method of example embodiment 113, wherein said circulating endothelial progenitor cells express CD47.
141. The method of example embodiment 113, wherein said circulating endothelial progenitor cells express CD35.
142. The method of example embodiment 113, wherein said circulating endothelial progenitor cells express DAF.
143. The method of example embodiment 113, wherein said circulating endothelial progenitor cells express CD73.
144. The method of example embodiment 113, wherein said circulating endothelial progenitor cells express CD90.
145. The method of example embodiment 113, wherein said circulating endothelial progenitor cells express CD105.
146. The method of example embodiment 113, wherein said circulating endothelial progenitor cells express aldehyde dehydrogenase.
147. The method of example embodiment 146, wherein said cells possess ability to inhibit an ongoing mixed lymphocyte reaction.
148. The method of example embodiment 147, wherein inhibition of said mixed lymphocyte reaction is quantified by assessment of proliferation of responding lymphocytes.
149. The method of example embodiment 148, wherein said responding lymphocytes are T cells.
150. The method of example embodiment 149, wherein said T cells are CD4 T cells.
151. The method of example embodiment 150, wherein said CD4 T cells are selected from a group comprising of: a) Th1; b) Th2; c) Th9; and d) Th17.
152. The method of example embodiment 151, wherein proliferation of Th1 T cells in said mixed lymphocyte reaction is inhibited by the cells of example embodiment 46.
153. The method of example embodiment 152, wherein said Th1 cells express STAT4 at a higher concentration than other CD4 T cells.
154. The method of example embodiment 152, wherein said Th1 cells express interleukin-2 at a higher concentration than other CD4 T cells.
155. The method of example embodiment 152, wherein said Th1 cells express interleukin-12 at a higher concentration than other CD4 T cells.
156. The method of example embodiment 152, wherein said Th1 cells express interleukin-15 at a higher concentration than other CD4 T cells.
157. The method of example embodiment 152, wherein said Th1 cells express interleukin-18 at a higher concentration than other CD4 T cells.
158. The method of example embodiment 152, wherein said Th1 cells express interferon gamma at a higher concentration than other CD4 T cells.
159. The method of example embodiment 151, wherein proliferation of Th9 T cells in said mixed lymphocyte reaction is inhibited by the cells of example embodiment 46.
160. The method of example embodiment 159, wherein said Th9 cells produce more interleukin-9 as compared to other CD4 T cells.
161. The method of example embodiment 151, wherein proliferation of Th17 T cells in said mixed lymphocyte reaction is inhibited by the cells of example embodiment 46.
162. The method of example embodiment 161, wherein said Th17 cells express more interleukin-17 as compared to other CD4 T cells.
163. The method of example embodiment 161, wherein said Th17 cells express more ror-gamma as compared to other CD4 T cells.
164. The method of example embodiment 161, wherein said Th17 cells express more NR2F6 as compared to other CD4 T cells.
165. The method of example embodiment 161, wherein said Th17 cells express more interleukin-17 receptor as compared to other CD4 T cells.
166. The method of example embodiment 161, wherein said Th17 cells express more interleukin-6 receptor as compared to other CD4 T cells.
167. The method of example embodiment 161, wherein said Th17 cells express more interleukin-21 receptor as compared to other CD4 T cells.
168. The method of example embodiment 161, wherein said Th17 cells express more interleukin-22 receptor as compared to other CD4 T cells.
169. The method of example embodiment 161, wherein said Th17 cells express more interleukin-23 receptor as compared to other CD4 T cells.
170. The method of example embodiment 161, wherein said Th17 cells express more interleukin-27 receptor as compared to other CD4 T cells.
171. The method of example embodiment 1, wherein an anti-inflammatory agent is administered together with said regenerative cells.
172. The method of example embodiment 171, wherein said anti-inflammatory agent a cytokine.
173. The method of example embodiment 172, wherein said cytokine is a Th2 cytokine.
174. The method of example embodiment 172, wherein said cytokine is a Th3 cytokine.
175. The method of example embodiment 172, wherein said cytokine is interleukin 1 receptor antagonist.
176. The method of example embodiment 172, wherein said cytokine is interleukin-3.
177. The method of example embodiment 172, wherein said cytokine is interleukin-4.
178. The method of example embodiment 172, wherein said cytokine is interleukin-7.
179. The method of example embodiment 172, wherein said cytokine is interleukin-10.
180. The method of example embodiment 172, wherein said cytokine is interleukin-13.
181. The method of example embodiment 172, wherein said cytokine is interleukin-14.
182. The method of example embodiment 172, wherein said cytokine is interleukin-16.
183. The method of example embodiment 172, wherein said cytokine is interleukin-20.
184. The method of example embodiment 172, wherein said cytokine is interleukin-35.
185. The method of example embodiment 172, wherein said cytokine is soluble HLA-G.
186. The method of example embodiment 172, wherein said cytokine is soluble ILT-3.
187. The method of example embodiment 172, wherein said cytokine is soluble ILT-4.
188. The method of example embodiment 172, wherein said cytokine is HGF-1.
189. The method of example embodiment 172, wherein said cytokine is angiopoietin.
190. The method of example embodiment 172, wherein said cytokine is VEGF.
191. The method of example embodiment 172, wherein said cytokine is IGF-1.
192. The method of example embodiment 172, wherein said cytokine is EGF-1.
193. The method of example embodiment 172, wherein said cytokine is Notch-1.
194. The method of example embodiment 172, wherein said cytokine is BDNF.
195. The method of example embodiment 172, wherein said cytokine is NGF-1.
196. The method of example embodiment 172, wherein said cytokine is FGF-1.
197. The method of example embodiment 172, wherein said cytokine is EGF-2.
198. The method of example embodiment 172, wherein said cytokine is PGE-2.
199. The method of example embodiment 172, wherein said cytokine is leukemia inhibitor factor.
200. The method of example embodiment 172, wherein said cytokine is placental growth factor.
201. The method of example embodiment 171, wherein said anti-inflammatory agent is an inhibitor of NF-kappa B.
202. The method of example embodiment 201, wherein said NF-kappa B inhibitor is Perrilyl alcohol.
203. The method of example embodiment 201, wherein said NF-kappa B inhibitor is Protein-bound polysaccharide from basidiomycetes.
204. The method of example embodiment 201, wherein said NF-kappa B inhibitor is Rocaglamides.
205. The method of example embodiment 201, wherein said NF-kappa B inhibitor is 15-deoxy-prostaglandin J(2).
206. The method of example embodiment 201, wherein said NF-kappa B inhibitor is Anandamide.
207. The method of example embodiment 201, wherein said NF-kappa B inhibitor is vestita.
208. The method of example embodiment 201, wherein said NF-kappa B inhibitor is Dehydroascorbic acid.
209. The method of example embodiment 201, wherein said NF-kappa B inhibitor is Herbimycin A.
210. The method of example embodiment 201, wherein said NF-kappa B inhibitor is Isorhapontigenin.
211. The method of example embodiment 201, wherein said NF-kappa B inhibitor is Manumycin A.
212. The method of example embodiment 201, wherein said NF-kappa B inhibitor is Pomegranate fruit extract.
213. The method of example embodiment 201, wherein said NF-kappa B inhibitor is Tetrandine.
214. The method of example embodiment 201, wherein said NF-kappa B inhibitor is Thienopyridine.
215. The method of example embodiment 201, wherein said NF-kappa B inhibitor is Acetyl-boswellic acids.
216. The method of example embodiment 201, wherein said NF-kappa B inhibitor is 1′-Acetoxychavicol acetate.
217. The method of example embodiment 201, wherein said NF-kappa B inhibitor is Apigenin.
218. The method of example embodiment 201, wherein said NF-kappa B inhibitor is Cardamomin.
219. The method of example embodiment 201, wherein said NF-kappa B inhibitor is Diosgenin.
220. The method of example embodiment 201, wherein said NF-kappa B inhibitor is Furonaphthoquinone.
221. The method of example embodiment 201, wherein said NF-kappa B inhibitor is Guggulsterone.
222. The method of example embodiment 201, wherein said NF-kappa B inhibitor is Falcarindol.
223. The method of example embodiment 201, wherein said NF-kappa B inhibitor is Honokiol.
224. The method of example embodiment 201, wherein said NF-kappa B inhibitor is Hypoestoxide.
225. The method of example embodiment 201, wherein said NF-kappa B inhibitor is Garcinone B.
226. The method of example embodiment 201, wherein said NF-kappa B inhibitor is Kahweol.
227. The method of example embodiment 201, wherein said NF-kappa B inhibitor is Kava.
228. The method of example embodiment 201, wherein said NF-kappa B inhibitor is mangostin.
229. The method of example embodiment 201, wherein said NF-kappa B inhibitor is mangostin.
230. The method of example embodiment 201, wherein said NF-kappa B inhibitor is N-acetylcysteine.
231. The method of example embodiment 201, wherein said NF-kappa B inhibitor is Piceatannol.
232. The method of example embodiment 201, wherein said NF-kappa B inhibitor is Plumbagin.
233. The method of example embodiment 201, wherein said NF-kappa B inhibitor is Quercetin.
234. The method of example embodiment 201, wherein said NF-kappa B inhibitor is Rosmarinic acid.
235. The method of example embodiment 201, wherein said NF-kappa B inhibitor is Staurosporine.
236. The method of example embodiment 201, wherein said NF-kappa B inhibitor is Sulforaphane.
237. The method of example embodiment 201, wherein said NF-kappa B inhibitor is phenylisothiocyanate.
238. The method of example embodiment 201, wherein said NF-kappa B inhibitor is Theaflavin.
239. The method of example embodiment 201, wherein said NF-kappa B inhibitor is Tilianin.
240. The method of example embodiment 201, wherein said NF-kappa B inhibitor is Tocotrienol.
241. The method of example embodiment 201, wherein said NF-kappa B inhibitor is Wedelolactone.
242. The method of example embodiment 201, wherein said NF-kappa B inhibitor is Withanolides.
243. The method of example embodiment 201, wherein said NF-kappa B inhibitor is Zerumbone.
244. The method of example embodiment 201, wherein said NF-kappa B inhibitor is Silibinin.
245. The method of example embodiment 201, wherein said NF-kappa B inhibitor is Betulinic acid.
246. The method of example embodiment 201, wherein said NF-kappa B inhibitor is Ursolic acid.
247. The method of example embodiment 201, wherein said NF-kappa B inhibitor is Monochloramine.
248. The method of example embodiment 201, wherein said NF-kappa B inhibitor is glycine chloramine.
249. The method of example embodiment 201, wherein said NF-kappa B inhibitor is Anethole.
250. The method of example embodiment 201, wherein said NF-kappa B inhibitor is B aoganning.
251. The method of example embodiment 201, wherein said NF-kappa B inhibitor is cyanidin 3-O-glucoside.
252. The method of example embodiment 201, wherein said NF-kappa B inhibitor is cyanidin 3-O-(2(G)-xylosylrutinoside.
253. The method of example embodiment 201, wherein said NF-kappa B inhibitor is cyanidin 3-O-rutinoside.
254. The method of example embodiment 201, wherein said NF-kappa B inhibitor is Buddlejasaponin IV.
255. The method of example embodiment 201, wherein said NF-kappa B inhibitor is Cacospongionolide B.
256. The method of example embodiment 201, wherein said NF-kappa B inhibitor is Calagualine.
257. The method of example embodiment 201, wherein said NF-kappa B inhibitor is Cardamonin.
258. The method of example embodiment 201, wherein said NF-kappa B inhibitor is Cycloepoxydon.
259. The method of example embodiment 201, wherein said NF-kappa B inhibitor is 1-hydroxy-2-hydroxymethyl-3-pent-1-enylbenzene.
260. The method of example embodiment 201, wherein said NF-kappa B inhibitor is Decursin.
261. The method of example embodiment 201, wherein said NF-kappa B inhibitor is Dexanabinol.
262. The method of example embodiment 201, wherein said NF-kappa B inhibitor is Digitoxin.
263. The method of example embodiment 201, wherein said NF-kappa B inhibitor is Diterpenes.
264. The method of example embodiment 201, wherein said NF-kappa B inhibitor is Docosahexaenoic acid.
265. The method of example embodiment 201, wherein said NF-kappa B inhibitor is oxidized low density lipoprotein.
266. The method of example embodiment 201, wherein said NF-kappa B inhibitor is 4-Hydroxynonenal (HNE).
267. The method of example embodiment 201, wherein said NF-kappa B inhibitor is Flavopiridol.
268. The method of example embodiment 201, wherein said NF-kappa B inhibitor is [6]-gingerol.
269. The method of example embodiment 201, wherein said NF-kappa B inhibitor is casparol.
270. The method of example embodiment 201, wherein said NF-kappa B inhibitor is Glossogyne tenuifolia.
271. The method of example embodiment 201, wherein said NF-kappa B inhibitor is inositol hexakisphosphate.
272. The method of example embodiment 201, wherein said NF-kappa B inhibitor is Phytic acid.
273. The method of example embodiment 201, wherein said NF-kappa B inhibitor is Prostaglandin A1.
274. The method of example embodiment 201, wherein said NF-kappa B inhibitor is 20(S)-Protopanaxatriol.
275. The method of example embodiment 201, wherein said NF-kappa B inhibitor is Rengyolone.
276. The method of example embodiment 201, wherein said NF-kappa B inhibitor is Rottlerin.
277. The method of example embodiment 201, wherein said NF-kappa B inhibitor is Saikosaponin-d.
278. The method of example embodiment 201, wherein said NF-kappa B inhibitor is Cacospongionolide B.
279. The method of example embodiment 201, wherein said NF-kappa B inhibitor is xenon.
280. The method of example embodiment 201, wherein said NF-kappa B inhibitor is argon.
281. The method of example embodiment 201, wherein said NF-kappa B inhibitor is radon.
282. The method of example embodiment 201, wherein said NF-kappa B inhibitor is ozone.
283. The method of example embodiment 201, wherein said NF-kappa B inhibitor is fish oil.
284. The method of example embodiment 201, wherein said NF-kappa B inhibitor is carbon monoxide.
285. The method of example embodiment 201, wherein said NF-kappa B inhibitor is decoy oligonucleotides.
286. The method of example embodiment 201, wherein said NF-kappa B inhibitor is antisense oligonucleotides.
287. The method of example embodiment 201, wherein said NF-kappa B inhibitor is short interfering RNA.
288. The method of example embodiment 201, wherein said NF-kappa B inhibitor is short hairpin RNA.
289. The method of example embodiment 201, wherein said NF-kappa B inhibitor is ribozyme based.
290. The method of example embodiment 201, wherein said NF-kappa B inhibitor is DNano based.
291. The method of example embodiment 201, wherein said NF-kappa B inhibitor is interleukin-10.
292. The method of example embodiment 201, wherein said NF-kappa B inhibitor is interleukin-2.
293. The method of example embodiment 201, wherein said NF-kappa B inhibitor is interleukin-3.
294. The method of example embodiment 201, wherein said NF-kappa B inhibitor is interleukin-8.
295. The method of example embodiment 201, wherein said NF-kappa B inhibitor is interleukin-9.
296. The method of example embodiment 201, wherein said NF-kappa B inhibitor is interleukin-12.
297. The method of example embodiment 201, wherein said NF-kappa B inhibitor is interleukin-15.
298. The method of example embodiment 201, wherein said NF-kappa B inhibitor is interleukin-17.
299. The method of example embodiment 201, wherein said NF-kappa B inhibitor is interleukin-20.
300. The method of example embodiment 201, wherein said NF-kappa B inhibitor is interleukin-33.
301. A method of in vivo regenerating organs in a post-mortem body comprising administration of regenerative cells and alternatively quantifying efficacy of said organ regeneration by assessment of organ functions and/or endothelial reactivity.
302. The method of example embodiment 301, wherein said regenerative cells are administered locally or systemically.
303. The method of example embodiment 302, wherein said regenerative cells are mesenchymal stem cell are derived from tissue comprising a group selected from: a) Wharton's Jelly; b) bone marrow; c) peripheral blood; d) mobilized peripheral blood; e) endometrium; f) hair follicle; g) deciduous tooth; h) testicle; i) adipose tissue; j) skin; k) amniotic fluid; l) cord blood; m) omentum; n) muscle; o) amniotic membrane; o) periventricular fluid; and p) placental tissue.
304. The method of example embodiment 303, wherein said mesenchymal stem cells express a marker or plurality of markers selected from a group comprising of: STRO-1, CD90, CD73, CD105, CD54, CD106, HLA-I markers, vimentin, ASMA, collagen-1, fibronectin, LFA-3, ICAM-1, PECAM-1, P-selectin, L-selectin, CD49b/CD29, CD49c/CD29, CD49d/CD29, CD61, CD18, CD29, thrombomodulin, telomerase, CD10, CD13, STRO-2, VCAM-1, CD146, and THY-1.
305. The method of example embodiment 304, wherein said mesenchymal stem cells do not express substantial levels of HLA-DR, CD117, and CD45.
306. The method of example embodiment 303, wherein said mesenchymal stem cells are generated from a pluripotent stem cell.
307. The method of example embodiment 306, wherein said pluripotent stem cell is selected from a group comprising of: a) an embryonic stem cell; b) an inducible pluripotent stem cell; c) a parthenogenic stem cell; and d) a somatic cell nuclear transfer derived stem cell.
308. The method of example embodiment 307, wherein said embryonic stem cell population expresses genes selected from a group comprising of: stage-specific embryonic antigens (SSEA) 3, SSEA 4, Tra-1-60 and Tra-1-81, Oct-3/4, Cripto, gastrin-releasing peptide (GRP) receptor, podocalyxin-like protein (PODXL), Rex-1, GCTM-2, Nanog, and human telomerase reverse transcriptase (hTERT).
309. The method of example embodiment 307, wherein said inducible pluripotent stem cell possesses markers selected from a group comprising of: CD10, CD13, CD44, CD73, CD90, PDGFr-alpha, PD-L2, and HLA-A,B,C and possesses ability to undergo at least 40 doublings in culture, while maintaining a normal karyotype upon passaging.
310. The method of example embodiment 307, wherein said parthenogenic stem cells wherein said parthenogenically derived stem cells are generated by addition of a calcium flux inducing agent to activate an oocyte followed by enrichment of cells expressing markers selected from a group comprising of SSEA-4, TRA 1-60 and TRA 1-81.
311. The method of example embodiment 307, wherein said somatic cell nuclear transfer derived stem cells possess a phenotype negative for SSEA-1 and positive for SSEA-3, SSEA-4, TRA-1-60, TRA-1-81, and alkaline phosphatase.
312. The method of example embodiment 306, wherein said mesenchymal stem cells are differentiated from a pluripotent stem cell source through culture in the presence of an inhibitor of the SMAD-2/3 pathway.
313. The method of example embodiment 312, wherein said mesenchymal stem cells are differentiated from a pluripotent stem cell source through culture in the presence of an inhibitor nucleic acid targeting the SMAD-2/3 pathway.
314. The method of example embodiment 313, wherein said nucleic acid inhibitor is selected from a group comprising of: a) an antisense oligonucleotide; b) a hairpin loop short interfering RNA; c) a chemically synthesized short interfering RNA molecule; and d) a hammerhead ribozyme.
315. The method of example embodiment 313, wherein said inhibitor of the SMAD-2/3 pathway is a small molecule inhibitor.
316. The method of example embodiment 315, wherein said small molecule inhibitor is SB-431542.
317. The method of example embodiment 306, wherein a selection process is used to enrich for mesenchymal stem cells differentiated from said pluripotent stem cell population.
318. The method of example embodiment 317, wherein said enrichment method comprises of positively selecting for cells expressing a marker associated with mesenchymal stem cells.
319. The method of example embodiment 318, wherein said marker of mesenchymal stem cells is selected from a group comprising of: STRO-1, CD90, CD73, CD105, CD54, CD106, HLA-I markers, vimentin, ASMA, collagen-1, fibronectin, LFA-3, ICAM-1, PECAM-1, P-selectin, L-selectin, CD49b/CD29, CD49c/CD29, CD49d/CD29, CD61, CD18, CD29, thrombomodulin, telomerase, CD10, CD13, STRO-2, VCAM-1, CD146, and THY-1.
320. The method of example embodiment 301, wherein the subject is treated to maintain hormonal homeostasis.
321. The method of example embodiment 320, wherein the subject is administered exogenous hormones and electrolyte support.
322. The method of any one of example embodiments 320-321, wherein the subject is maintained alive by means of life support.
323. The method of any one of example embodiments 320-322, wherein the regenerative cells are prepared by administering to the subject an agent to mobilize regenerative cells from bone marrow into peripheral blood of the subject; and isolating said regenerative cells from peripheral blood of the subject.
324. The method of any one of example embodiments 320-323, wherein the agent to mobilize regenerative cells is granulocyte colony-stimulating factor (G-CSF).
325. The method of any one of example embodiments 320-323, wherein the agent to mobilize regenerative cells is granulocyte monocyte colony-stimulating factor (GM-CSF).
326. The method of any one of example embodiments 320-323, wherein the agent to mobilize regenerative cells is Leukemia Inhibiting Factor (LIF).
327. The method of any one of example embodiments 320-323, wherein the agent to mobilize regenerative cells is HGF-1.
328. The method of any one of example embodiments 320-323, wherein the agent to mobilize regenerative cells is FGF-1.
329. The method of any one of example embodiments 320-323, wherein the agent to mobilize regenerative cells is FGF-2.
330. The method of any one of example embodiments 320-323, wherein the agent to mobilize regenerative cells is AMD-3100.
331. The method of any one of example embodiments 320-323, wherein the agent to mobilize regenerative cells is M=CSF.
332. The method of any one of example embodiments 320-323, wherein the agent to mobilize regenerative cells is ozone therapy.
333. The method of any one of example embodiments 320-323, wherein the agent to mobilize regenerative cells is IL-2.
334. The method of any one of example embodiments 320-323, wherein the agent to mobilize regenerative cells is FLT-3 ligand.
335. The method of any one of example embodiments 320-323, wherein the agent to mobilize regenerative cells is TNF alpha.
336. The method of any one of example embodiments 320-323, wherein the agent to mobilize regenerative cells is hCG.
337. The method of any one of example embodiments 320-323, wherein the agent to mobilize regenerative cells is hyperbaric oxygen.
338. The method of any one of example embodiments 320-323, wherein the agent to mobilize regenerative cells is BDNF.
339. The method of any one of example embodiments 320-323, wherein the agent to mobilize regenerative cells is NGF-1.
340. The method of any one of example embodiments 320-323, wherein the agent to mobilize regenerative cells is VEGF.
341. The method of any one of example embodiments 320-323, wherein the regenerative cells are isolated from peripheral circulation of the subject by apheresis using an antibody that has selective affinity to said regenerative cells.
342. The method of example embodiment 341, wherein said regenerative cells are isolated with an antibody capable of binding cells expressing CD31.
343. The method of example embodiment 341, wherein said regenerative cells are isolated with an antibody capable of binding cells expressing CD33.
344. The method of example embodiment 341, wherein said regenerative cells are isolated with an antibody capable of binding cells expressing CD34.
345. The method of example embodiment 341, wherein said regenerative cells are isolated with an antibody capable of binding cells expressing CD133.
346. The method of example embodiment 341, wherein said regenerative cells are isolated with an antibody capable of binding cells expressing CD34.
347. The method of example embodiment 341, wherein said regenerative cells are isolated with an antibody capable of binding cells expressing CD31 and CD34.
348. The method of example embodiment 341, wherein said regenerative cells are isolated with an antibody capable of binding cells expressing CD31 and CD33.
349. The method of example embodiment 341, wherein said regenerative cells are isolated with an antibody capable of binding cells expressing CD31 and VEGF receptor.
350. The method of example embodiment 341, wherein said regenerative cells are isolated with an antibody capable of binding cells expressing CD31 and HGF-1 receptor.
351. The method of example embodiment 341, wherein said regenerative cells are isolated with an antibody capable of binding cells expressing CD31 and CD107.
352. The method of example embodiment 341, wherein said regenerative cells are isolated with an antibody capable of binding cells expressing CD31 and stem cell factor.
353. The method of example embodiment 341, wherein said regenerative cells are isolated with an antibody capable of binding cells expressing TLR-4 and CD34.
354. The method of example embodiment 341, wherein said regenerative cells are isolated with an antibody capable of binding cells expressing TLR-4 and CD33.
355. The method of example embodiment 341, wherein said regenerative cells are isolated with an antibody capable of binding cells expressing TLR-4 and VEGF receptor.
356. The method of example embodiment 341, wherein said regenerative cells are isolated with an antibody capable of binding cells expressing TLR-4 and HGF-1 receptor.
357. The method of example embodiment 341, wherein said regenerative cells are isolated with an antibody capable of binding cells expressing TLR-4 and stem cell factor.
358. The method of example embodiment 341, wherein said regenerative cells are isolated with an antibody capable of binding cells expressing TLR-4 and CD90.
359. The method of example embodiment 341, wherein said regenerative cells are isolated with an antibody capable of binding cells expressing TLR-4 and CD13.
360. The method of example embodiment 341, wherein said regenerative cells are isolated with an antibody capable of binding cells expressing TLR-4 and CD29.
361. The method of example embodiment 341, wherein said regenerative cells are isolated with an antibody capable of binding cells expressing TLR-4 and CD44.
362. The method of example embodiment 341, wherein said regenerative cells are isolated with an antibody capable of binding cells expressing TLR-4 and CD71.
363. The method of example embodiment 341, wherein said regenerative cells are isolated with an antibody capable of binding cells expressing TLR-4 and CD73.
364. The method of example embodiment 341, wherein said regenerative cells are isolated with an antibody capable of binding cells expressing TLR-4 and CD105.
365. The method of example embodiment 341, wherein said regenerative cells are isolated with an antibody capable of binding cells expressing TLR-4 and CD166.
366. The method of example embodiment 341, wherein said regenerative cells are isolated with an antibody capable of binding cells expressing TLR-4 and STRO-1.
367. The method of example embodiment 341, wherein said regenerative cells are isolated with an antibody capable of binding cells expressing TLR-4 and STRO-4.
368. The method of example embodiment 341, wherein said regenerative cells are isolated with an antibody capable of binding cells expressing TLR-4 and TNF receptor p55.
369. The method of example embodiment 341, wherein said regenerative cells are isolated with an antibody capable of binding cells expressing TLR-4 and TNF receptor p75.
370. The method of example embodiment 341, wherein said regenerative cells are isolated with an antibody capable of binding cells expressing TLR-4 and CD227.
371. The method of example embodiment 341, wherein said regenerative cells are isolated with an antibody capable of binding cells expressing c-kit and CD34.
372. The method of example embodiment 341, wherein said regenerative cells are isolated with an antibody capable of binding cells expressing c-kit and CD33.
373. The method of example embodiment 341, wherein said regenerative cells are isolated with an antibody capable of binding cells expressing c-kit and VEGF receptor.
374. The method of example embodiment 341, wherein said regenerative cells are isolated with an antibody capable of binding cells expressing c-kit and HGF-1 receptor.
375. The method of example embodiment 341, wherein said regenerative cells are isolated with an antibody capable of binding cells expressing c-kit and stem cell factor.
376. The method of example embodiment 341, wherein said regenerative cells are isolated with an antibody capable of binding cells expressing c-kit and CD90.
377. The method of example embodiment 341, wherein said regenerative cells are isolated with an antibody capable of binding cells expressing c-kit and CD13.
378. The method of example embodiment 341, wherein said regenerative cells are isolated with an antibody capable of binding cells expressing c-kit and CD29.
379. The method of example embodiment 341, wherein said regenerative cells are isolated with an antibody capable of binding cells expressing c-kit and CD44.
380. The method of example embodiment 341, wherein said regenerative cells are isolated with an antibody capable of binding cells expressing c-kit and CD71.
381. The method of example embodiment 341, wherein said regenerative cells are isolated with an antibody capable of binding cells expressing c-kit and CD73.
382. The method of example embodiment 341, wherein said regenerative cells are isolated with an antibody capable of binding cells expressing c-kit and CD105.
383. The method of example embodiment 341, wherein said regenerative cells are isolated with an antibody capable of binding cells expressing c-kit and CD166.
384. The method of example embodiment 341, wherein said regenerative cells are isolated with an antibody capable of binding cells expressing c-kit and STRO-1.
385. The method of example embodiment 341, wherein said regenerative cells are isolated with an antibody capable of binding cells expressing c-kit and STRO-4.
386. The method of example embodiment 341, wherein said regenerative cells are isolated with an antibody capable of binding cells expressing c-kit and TNF receptor p55.
387. The method of example embodiment 341, wherein said regenerative cells are isolated with an antibody capable of binding cells expressing c-kit and TNF receptor p75.
388. The method of example embodiment 341, wherein said regenerative cells are isolated with an antibody capable of binding cells expressing c-kit and CD227.
389. The method of example embodiment 341, wherein said regenerative cells are isolated with an antibody capable of binding cells expressing OCT-4 and CD34.
390. The method of example embodiment 341, wherein said regenerative cells are isolated with an antibody capable of binding cells expressing OCT-4 and CD33.
391. The method of example embodiment 341, wherein said regenerative cells are isolated with an antibody capable of binding cells expressing OCT-4 and VEGF receptor.
392. The method of example embodiment 341, wherein said regenerative cells are isolated with an antibody capable of binding cells expressing OCT-4 and HGF-1 receptor.
393. The method of example embodiment 341, wherein said regenerative cells are isolated with an antibody capable of binding cells expressing OCT-4 and stem cell factor.
394. The method of example embodiment 341, wherein said regenerative cells are isolated with an antibody capable of binding cells expressing OCT-4 and CD90.
395. The method of example embodiment 341, wherein said regenerative cells are isolated with an antibody capable of binding cells expressing OCT-4 and CD13.
396. The method of example embodiment 341, wherein said regenerative cells are isolated with an antibody capable of binding cells expressing OCT-4 and CD29.
397. The method of example embodiment 341, wherein said regenerative cells are isolated with an antibody capable of binding cells expressing OCT-4 and CD44.
398. The method of example embodiment 341, wherein said regenerative cells are isolated with an antibody capable of binding cells expressing OCT-4 and CD71.
399. The method of example embodiment 341, wherein said regenerative cells are isolated with an antibody capable of binding cells expressing OCT-4 and CD73.
400. A method of preventing or treating organ damage in a deceased individual by administration of regenerative cells.
401. The method of example embodiment 400, wherein said regenerative cells are mesenchymal stem cells.
402. The method of example embodiment 401, wherein said mesenchymal stem cells are naturally occurring mesenchymal stem cells.
403. The method of example embodiment 401, wherein said mesenchymal stem cells are generated in vitro.
404. The method of example embodiment 402, wherein said naturally occurring mesenchymal stem cells are tissue derived.
405. The method of example embodiment 402, wherein said naturally occurring mesenchymal stem cells are derived from a bodily fluid.
406. The method of example embodiment 404, wherein said tissue derived mesenchymal stem cells are selected from a group comprising of: a) bone marrow; b) perivascular tissue; c) adipose tissue; d) placental tissue; e) amniotic membrane; f) omentum; g) tooth; h) umbilical cord tissue; i) fallopian tube tissue; j) hepatic tissue; k) renal tissue; l) cardiac tissue; m) tonsillar tissue; n) testicular tissue; o) ovarian tissue; p) neuronal tissue; q) auricular tissue; r) colonic tissue; s) submucosal tissue; t) hair follicle tissue; u) pancreatic tissue; v) skeletal muscle tissue; and w) subepithelial umbilical cord tissue.
407. The method of example embodiment 404, wherein said tissue derived mesenchymal stem cells are isolated from tissues containing cells selected from a group of cells comprising of: endothelial cells, epithelial cells, dermal cells, endodermal cells, mesodermal cells, fibroblasts, osteocytes, chondrocytes, natural killer cells, dendritic cells, hepatic cells, pancreatic cells, stromal cells, salivary gland mucous cells, salivary gland serous cells, von Ebner's gland cells, mammary gland cells, lacrimal gland cells, ceruminous gland cells, eccrine sweat gland dark cells, eccrine sweat gland clear cells, apocrine sweat gland cells, gland of Moll cells, sebaceous gland cells. bowman's gland cells, Brunner's gland cells, seminal vesicle cells, prostate gland cells, bulbourethral gland cells, Bartholin's gland cells, gland of Littre cells, uterus endometrium cells, isolated goblet cells, stomach lining mucous cells, gastric gland zymogenic cells, gastric gland oxyntic cells, pancreatic acinar cells, paneth cells, type II pneumocytes, clara cells, somatotropes, lactotropes, thyrotropes, gonadotropes, corticotropes, intermediate pituitary cells, magnocellular neurosecretory cells, gut cells, respiratory tract cells, thyroid epithelial cells, parafollicular cells, parathyroid gland cells, parathyroid chief cell, oxyphil cell, adrenal gland cells, chromaffin cells, Leydig cells, theca interna cells, corpus luteum cells, granulosa lutein cells, theca lutein cells, juxtaglomerular cell, macula densa cells, peripolar cells, mesangial cell, blood vessel and lymphatic vascular endothelial fenestrated cells, blood vessel and lymphatic vascular endothelial continuous cells, blood vessel and lymphatic vascular endothelial splenic cells, synovial cells, serosal cell (lining peritoneal, pleural, and pericardial cavities), squamous cells, columnar cells, dark cells, vestibular membrane cell (lining endolymphatic space of ear), stria vascularis basal cells, stria vascularis marginal cell (lining endolymphatic space of ear), cells of Claudius, cells of Boettcher, choroid plexus cells, pia-arachnoid squamous cells, pigmented ciliary epithelium cells, nonpigmented ciliary epithelium cells, corneal endothelial cells, peg cells, respiratory tract ciliated cells, oviduct ciliated cell, uterine endometrial ciliated cells, rete testis ciliated cells, ductulus efferens ciliated cells, ciliated ependymal cells, epidermal keratinocytes, epidermal basal cells, keratinocyte of fingernails and toenails, nail bed basal cells, medullary hair shaft cells, cortical hair shaft cells, cuticular hair shaft cells, cuticular hair root sheath cells, hair root sheath cells of Huxley's layer, hair root sheath cells of Henle's layer, external hair root sheath cells, hair matrix cells, surface epithelial cells of stratified squamous epithelium, basal cell of epithelia, urinary epithelium cells, auditory inner hair cells of organ of Corti, auditory outer hair cells of organ of Corti, basal cells of olfactory epithelium, cold-sensitive primary sensory neurons, heat-sensitive primary sensory neurons, Merkel cells of epidermis, olfactory receptor neurons, pain-sensitive primary sensory neurons, photoreceptor rod cells, photoreceptor blue-sensitive cone cells, photoreceptor green-sensitive cone cells, photoreceptor red-sensitive cone cells, proprioceptive primary sensory neurons, touch-sensitive primary sensory neurons, type I carotid body cells, type II carotid body cell (blood pH sensor), type I hair cell of vestibular apparatus of ear (acceleration and gravity), type II hair cells of vestibular apparatus of ear, type I taste bud cells cholinergic neural cells, adrenergic neural cells, peptidergic neural cells, inner pillar cells of organ of Corti, outer pillar cells of organ of Corti, inner phalangeal cells of organ of Corti, outer phalangeal cells of organ of Corti, border cells of organ of Corti, Hensen cells of organ of Corti, vestibular apparatus supporting cells, taste bud supporting cells, olfactory epithelium supporting cells, Schwann cells, satellite cells, enteric glial cells, astrocytes, neurons, oligodendrocytes, spindle neurons, anterior lens epithelial cells, crystallin-containing lens fiber cells, hepatocytes, adipocytes, white fat cells, brown fat cells, liver lipocytes, kidney glomerulus parietal cells, kidney glomerulus podocytes, kidney proximal tubule brush border cells, loop of Henle thin segment cells, kidney distal tubule cells, kidney collecting duct cells, type I pneumocytes, pancreatic duct cells, nonstriated duct cells, duct cells, intestinal brush border cells, exocrine gland striated duct cells, gall bladder epithelial cells, ductulus efferens nonciliated cells, epididymal principal cells, epididymal basal cells, ameloblast epithelial cells, planum semilunatum epithelial cells, organ of Corti interdental epithelial cells, loose connective tissue fibroblasts, corneal keratocytes, tendon fibroblasts, bone marrow reticular tissue fibroblasts, nonepithelial fibroblasts, pericytes, nucleus pulposus cells, cementoblast/cementocytes, odontoblasts, odontocytes, hyaline cartilage chondrocytes, fibrocartilage chondrocytes, elastic cartilage chondrocytes, osteoblasts, osteocytes, osteoclasts, osteoprogenitor cells, hyalocytes, stellate cells (ear), hepatic stellate cells (Ito cells), pancreatic stelle cells, red skeletal muscle cells, white skeletal muscle cells, intermediate skeletal muscle cells, nuclear bag cells of muscle spindle, nuclear chain cells of muscle spindle, satellite cells, ordinary heart muscle cells, nodal heart muscle cells, Purkinje fiber cells, smooth muscle cells, myoepithelial cells of iris, myoepithelial cell of exocrine glands, melanocytes, retinal pigmented epithelial cells, oogonia/oocytes, spermatids, spermatocytes, spermatogonium cells, spermatozoa, ovarian follicle cells, Sertoli cells, thymus epithelial cell, and/or interstitial kidney cells.
408. The method of example embodiment 401, wherein said mesenchymal stem cells are plastic adherent.
409. The method of example embodiment 401, wherein said mesenchymal stem cells express a marker selected from a group comprising of: a) CD73; b) CD90; and c) CD105.
410. The method of example embodiment 401, wherein said mesenchymal stem cells lack expression of a marker selected from a group comprising of: a) CD14; b) CD45; and c) CD34.
411. The method of example embodiment 406, wherein said mesenchymal stem cells from umbilical cord tissue express markers selected from a group comprising of; a) oxidized low density lipoprotein receptor 1, b) chemokine receptor ligand 3; and c) granulocyte chemotactic protein.
412. The method of example embodiment 406, wherein said mesenchymal stem cells from umbilical cord tissue do not express markers selected from a group comprising of: a) CD117; b) CD31; c) CD34; and CD45;
413. The method of example embodiment 406, wherein said mesenchymal stem cells from umbilical cord tissue express, relative to a human fibroblast, increased levels of interleukin 8 and reticulon 1
414. The method of example embodiment 406, wherein said mesenchymal stem cells from umbilical cord tissue have the potential to differentiate into cells of at least a skeletal muscle, vascular smooth muscle, pericyte or vascular endothelium phenotype.
415. The method of example embodiment 406, wherein said mesenchymal stem cells from umbilical cord tissue express markers selected from a group comprising of: a) CD10; b) CD13; c) CD44; d) CD73; and e) CD90.
416. The method of example embodiment 406, wherein said umbilical cord tissue mesenchymal stem cell is an isolated umbilical cord tissue cell isolated from umbilical cord tissue substantially free of blood that is capable of self-renewal and expansion in culture,
417. The method of example embodiment 416, wherein said umbilical cord tissue mesenchymal stem cells has the potential to differentiate into cells of other phenotypes.
418. The method of example embodiment 417, wherein said other phenotypes comprise: a) osteocytic; b) adipogenic; and c) chondrogenic differentiation.
419. The method of example embodiment 406, wherein said cord tissue derived mesenchymal stem cells can undergo at least 20 doublings in culture.
420. The method of example embodiment 406, wherein said cord tissue derived mesenchymal stem cell maintains a normal karyotype upon passaging
421. The method of example embodiment 406, wherein said cord tissue derived mesenchymal stem cell expresses a marker selected from a group of markers comprised of: a) CD10 b) CD13; c) CD44; d) CD73; e) CD90; f) PDGFr-alpha; g) PD-L2; and h) HLA-A,B,C
422. The method of example embodiment 406, wherein said cord tissue mesenchymal stem cells does not express one or more markers selected from a group comprising of; a) CD31; b) CD34; c) CD45; d) CD80; e) CD86; f) CD117; g) CD141; h) CD178; i) B7-H2; j) HLA-G and k) HLA-DR,DP,DQ.
423. The method of example embodiment 406, wherein said umbilical cord tissue-derived cell secretes factors selected from a group comprising of: a) MCP-1; b) MIP1beta; c) IL-6; d) IL-8; e) GCP-2; f) HGF; g) KGF; h) FGF; i) HB-EGF; j) BDNF; k) TPO; 1) RANTES; and m) TIMP1
424. The method of example embodiment 406, wherein said umbilical cord tissue derived cells express markers selected from a group comprising of: a) TRA1-60; b) TRA1-81; c) SSEA3; d) SSEA4; and e) NANOG.
425. The method of example embodiment 406, wherein said umbilical cord tissue-derived cells are positive for alkaline phosphatase staining.
426. The method of example embodiment 406, wherein said umbilical cord tissue-derived cells are capable of differentiating into one or more lineages selected from a group comprising of; a) ectoderm; b) mesoderm, and; c) endoderm.
427. The method of example embodiment 406, wherein said bone marrow derived mesenchymal stem cells possess markers selected from a group comprising of: a) CD73; b) CD90; and c) CD105.
428. The method of example embodiment 406, wherein said bone marrow derived mesenchymal stem cells possess markers selected from a group comprising of: a) LFA-3; b) ICAM-1; c) PECAM-1; d) P-selectin; e) L-selectin; f) CD49b/CD29; g) CD49c/CD29; h) CD49d/CD29; i) CD29; j) CD18; k) CD61; l) 6-19; m) thrombomodulin; n) telomerase; o) CD10; p) CD13; and q) integrin beta.
429. The method of example embodiment 406, wherein said bone marrow derived mesenchymal stem cell is a mesenchymal stem cell progenitor cell.
430. The method of example embodiment 429, wherein said mesenchymal progenitor cells are a population of bone marrow mesenchymal stem cells enriched for cells containing STRO-1
431. The method of example embodiment 430, wherein said mesenchymal progenitor cells express both STRO-1 and VCAM-1.
432. A method of example embodiment 430, wherein said STRO-1 expressing cells are negative for at least one marker selected from the group consisting of: a) CBFA-1; b) collagen type II; c) PPAR.gamma2; d) osteopontin; e) osteocalcin; f) parathyroid hormone receptor; g) leptin; h) H-ALBP; i) aggrecan; j) Ki67, and k) glycophorin A.
433. The method of example embodiment 406, wherein said bone marrow mesenchymal stem cells lack expression of CD14, CD34, and CD45.
434. The method of example embodiment 432, wherein said STRO-1 expressing cells are positive for a marker selected from a group comprising of: a) VACM-1; b) TKY-1; c) CD146 and; d) STRO-2
435. The method of example embodiment 406, wherein said bone marrow mesenchymal stem cell express markers selected from a group comprising of: a) CD13; b) CD34; c) CD56 and; d) CD117
436. The method of example embodiment 435, wherein said bone marrow mesenchymal stem cells do not express CD10.
437. The method of example embodiment 435, wherein said bone marrow mesenchymal stem cells do not express CD2, CD5, CD14, CD19, CD33, CD45, and DRII.
438. The method of example embodiment 435, wherein said bone marrow mesenchymal stem cells express CD13, CD34, CD56, CD90, CD117 and nestin, and which do not express CD2, CD3, CD10, CD14, CD16, CD31, CD33, CD45 and CD64.
439. The method of example embodiment 406, wherein said skeletal muscle stem cells express markers selected from a group comprising of: a) CD13; b) CD34; c) CD56 and; d) CD117
440. The method of example embodiment 440, wherein said skeletal muscle mesenchymal stem cells do not express CD10.
441. The method of example embodiment 440, wherein said skeletal muscle mesenchymal stem cells do not express CD2, CD5, CD14, CD19, CD33, CD45, and DRII.
442. The method of example embodiment 440, wherein said bone marrow mesenchymal stem cells express CD13, CD34, CD56, CD90, CD117 and nestin, and which do not express CD2, CD3, CD10, CD14, CD16, CD31, CD33, CD45 and CD64.
443. The method of example embodiment 406, wherein said subepithelial umbilical cord derived mesenchymal stem cells possess markers selected from a group comprising of; a) CD29; b) CD73; c) CD90; d) CD166; e) SSEA4; f) CD9; g) CD44; h) CD146; and i) CD105
444. The method of example embodiment 443, wherein said subepithelial umbilical cord derived mesenchymal stem cells do not express markers selected from a group comprising of; a) CD45; b) CD34; c) CD14; d) CD79; e) CD106; f) CD86; g) CD80; h) CD19; i) CD117; j) Stro-1 and k) HLA-DR.
445. The method of example embodiment 443, wherein said subepithelial umbilical cord derived mesenchymal stem cells express CD29, CD73, CD90, CD166, SSEA4, CD9, CD44, CD146, and CD105.
446. The method of example embodiment 143, wherein said subepithelial umbilical cord derived mesenchymal stem cells do not express CD45, CD34, CD14, CD79, CD106, CD86, CD80, CD19, CD117, Stro-1, and HLA-DR.
447. The method of example embodiment 443, wherein said subepithelial umbilical cord derived mesenchymal stem cells are positive for SOX2.
448. The method of example embodiment 443, wherein said subepithelial umbilical cord derived mesenchymal stem cells are positive for OCT4.
449. The method of example embodiment 443, wherein said subepithelial umbilical cord derived mesenchymal stem cells are positive for OCT4 and SOX2.
450. A method of increasing the quality of organs for transplantation comprising the steps of: a) obtaining a brain-dead patient; b) maintaining viability of said brain dead patient by one or more life supporting technologies; c) connecting circulation of said brain dead patient to an extracorporeal device containing regenerative and/or immune cells; d) allowing said extracorporeal device to provide regenerative and/or immune modulatory factors to said brain dead patient; e) assessing regeneration of said organ and f) when appropriate harvesting said organ.
451. The method of example embodiment 450, wherein said extracorporeal device a) provides an extracorporeal circuit; b) places regenerative cells in said extracorporeal circuit in a manner such that regenerative cells are in contact with circulation of said patient's non-cellular component of blood; c) ensures said circuit blocks entrance of said regenerative cells from said extracorporeal means into said patient blood; and d) allows soluble factors produced by said regenerative cells to enter circulation of said patient.
452. The method of example embodiment 451, wherein said extracorporeal circuit is similar to a dialysis circuit.
453. The method of example embodiment 451, wherein said extracorporeal circuit is an extracorporeal bioreactor, wherein said extracorporeal bioreactor comprises a compartment comprising regenerative cells and a selectively permeable membrane in contact with the cells that does not permit passage of said cells and which permits passage of regenerative factors in the bodily fluid of the patient, said regenerative cells capable of secreting said regenerative factors at a basal rate or at an inducible rate, depending on the needs of the patient.
454. The method of example embodiment 451, wherein said regenerative cells are amniotic fluid stem cells.
455. The method of example embodiment 451, wherein said amniotic fluid stem cells are characterized by the following cell surface markers: SSEA3, SSEA4, Tra-1-60, Tra-1-81, Tra-2-54, HLA class I, CD13, CD44, CD49b, and CD105.
456. The method of example embodiment 451, wherein said amniotic fluid stem cells are distinguished by the absence of the antigen markers CD34, CD45, and HLA Class II.
467. The method of example embodiment 454, wherein said amniotic fluid stem cells are cultured prior to use in said extracorporeal device.
468. The method of example embodiment 457, wherein said amniotic fluid stem cells are cultured for at least 14 days.
469. The method of example embodiment 457, wherein said amniotic fluid stem cells are maintained in an undifferentiated state during said culture.
470. The method of example embodiment 457, wherein said amniotic fluid stem cells are cultured in the presence of nerve growth factor), bFGF, dibutryl cAMP, IBMX, and/or retinoic acid) for four weeks.
471. The method of example embodiment 454, wherein said amniotic fluid stem cells are activated before administration.
472. The method of example embodiment 461, wherein said activation involves pretreatment with a cytokine.
473. The method of example embodiment 462, wherein said pretreatment with cytokines induces upregulation of complement inhibitory molecules.
474. The method of example embodiment 463, wherein said complement inhibitory molecule comprises of CD35.
475. The method of example embodiment 463, wherein said complement inhibitory molecule comprises of CD46.
476. The method of example embodiment 463, wherein said complement inhibitory molecule comprises of C4BP.
477. The method of example embodiment 463, wherein said complement inhibitory molecule comprises of CD55.
478. The method of example embodiment 463, wherein said complement inhibitory molecule comprises of Factor H.
479. The method of claim 463, wherein said cytokine is interleukin-1.
480. The method of claim 463, wherein said interleukin-1 is administered to said cells at a concentration of 1-100 nanograms per milliliter of tissue culture media.
481. The method of claim 463, wherein said interleukin-1 is administered to said cells at a concentration of 20-40 nanograms per milliliter of tissue culture media.
482. The method of claim 463, wherein said interleukin-1 is administered to said cells at a concentration of 30 nanograms per milliliter of tissue culture media.
483. The method of claim 463, wherein cytokine is interferon gamma.
484. The method of claim 481, wherein said interferon gamma is administered to said cells at a concentration of 1-1000 IU of interferon gamma per ml of tissue culture media.
485. The method of claim 481, wherein said interferon gamma is administered to said cells at a concentration of 100-500 IU of interferon gamma per ml of tissue culture media.
486. The method of claim 481, wherein said interferon gamma is administered to said cells at a concentration of 250 IU of interferon gamma per ml of tissue culture media.

What is claimed is:

1. A method of increasing the quality of organs for transplantation comprising the steps of:
a) obtaining a brain-dead patient;
b) maintaining viability of said brain dead patient by one or more life supporting technologies;
c) administering to said patient one or more regenerative cell populations and;
d) harvesting said organs.

2. The method of claim 1, wherein said regenerative cell is a stem cell, including possibility a pluripotent stem cell or a mesenchymal stem cell.

3. The method of claim 2, wherein said pluripotent stem cells are selected from a group of cells comprising:
a) inducible pluripotent stem cells;
b) somatic cell nuclear transfer derived stem cells;
c) embryonic stem cells; and
d) parthenogenic derived stem cells.

4. The method of claim 2, wherein said pluripotent stem cells are exposed to inflammatory stress before being provided to the brain dead patient.

5. The method of claim 4, wherein said inflammatory stress is exposure to a toll-like receptor.

6. The method of claim 3, wherein said inducible pluripotent stem cell possesses markers selected from a group comprising of: CD10, CD13, CD44, CD73, CD90, PDGFr-alpha, PD-L2, and HLA-A,B,C and possesses ability to undergo at least 40 doublings in culture, while maintaining a normal karyotype upon passaging.

7. The method of claim 6, wherein said inducible pluripotent stem cells express OCT4.

8. The method of claim 3, wherein said parthenogenic stem cells wherein said parthenogenically derived stem cells are generated by addition of a calcium flux inducing agent to activate an oocyte followed by enrichment of cells expressing markers selected from a group comprising of SSEA-4, TRA 1-60 and TRA 1-81.

9. The method of claim 3, wherein said somatic cell nuclear transfer derived stem cells possess a phenotype negative for SSEA-1 and positive for SSEA-3, SSEA-4, TRA-1-60, TRA-1-81, and alkaline phosphatase.

10. The method of claim 3, wherein said mesenchymal stem cell are derived from tissue selected from the group consisting of a) Wharton's Jelly; b) bone marrow; c) peripheral blood; d) mobilized peripheral blood; e) endometrium; f) hair follicle; g) deciduous tooth; h) testicle; i) adipose tissue; j) skin; k) amniotic fluid; l) cord blood; m) omentum; n) muscle; o) amniotic membrane; o) periventricular fluid; and p) placental tissue.

11. The method of claim 10, wherein said mesenchymal stem cells express a marker or plurality of markers selected from the group consisting of: STRO-1, CD90, CD73, CD105, CD54, CD106, HLA-I markers, vimentin, ASMA, collagen-1, fibronectin, LFA-3, ICAM-1, PECAM-1, P-selectin, L-selectin, CD49b/CD29, CD49c/CD29, CD49d/CD29, CD61, CD18, CD29, thrombomodulin, telomerase, CD10, CD13, STRO-2, VCAM-1, CD146, and THY-1.

12. The method of claim 11, wherein said mesenchymal stem cells do not express substantial levels of HLA-DR, CD117, and CD45.

13. The method of claim 11, wherein said mesenchymal stem cells express CD56.

14. The method of claim 11, wherein said mesenchymal stem cell are activated by exposure to a toll like receptor agonist.

15. The method of claim 1, wherein said regenerative cells are monocytes.

16. The method of claim 15, wherein said regenerative cells are monocytes that have been treated with interleukin-10.

17. The method of claim 15, wherein said regenerative cells are monocytes that have been exposed to hypoxia.

18. The method of claim 15, wherein said regenerative cells are monocytes that have been exposed to HGF-1.

19. The method of claim 15, wherein said regenerative cells are monocytes that have been exposed to FGF-1.

20. The method of claim 15, wherein said regenerative cells are monocytes that have been exposed to krypton.
